(19) United States
(12) Patent Application Publication
(10) Pub. No.: US 2006/0235428 A1
(43) Pub. Date: Oct. 19, 2006

(54) Ocular inlay with locator

(76) Inventor: Thomas A. Silvestrini, Alamo, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/106,040

(22) Filed: Apr. 14, 2005

(51) Int. Cl. A61F 9/013; A61F 2/14

(52) U.S. Cl. 606/107; 623/906

(57) ABSTRACT

A therapeutic ocular inlay for treatment of one or more vision defects including a locator structure or depth marker to facilitate location of the inlay post-implant. The locator structure or marker extends at least partially outside a pupil region. The depth or layer at which the inlay is implanted can be determined by locating the depth marker without requiring incisions with the pupil region. The locator structure can be substantially invisible under casual observation, however can be provided with enhanced optical contrast characteristics under selected viewing conditions. The locator is thus unobtrusive, however can be readily located in a clinical setting, for example with artificial UV illumination. The locator can be connected to, e.g., integrally formed with, the inlay or may be a separate component provided in combination with the inlay.

### BACKGROUND OF THE INVENTION

1. Field of the Invention

This application relates to ocular devices and, more particularly, to corneal inlays and other ocular implant devices with an unobtrusive locator structure that indicates the location of (e.g., the depth of) the ocular device within the eye when implanted.

2. Description of the Related Art

The healthy human eye is capable of receiving incident light from a range of viewing distances and appropriately refracting the incident light for proper focus at the retina such that the person has a clear view of the objects. The cornea and crystalline lens interact in a cooperative manner to provide this refraction. The healthy eye adjusts its refractive characteristics to provide the person with clear focused vision of objects both near and distant. This adjustment is normally provided by ciliary muscles attached to the lens that change the shape of the lens when the muscles flex. This process of adjusting is commonly referred to as accommodation.

Many people suffer one or more vision defects that impair accommodation. For example, presbyopia is a fairly common condition in people over age 40 that compromises the ability to accommodate for near objects. Presbyopia is experienced as a blurring of the person's vision and frequently arises when the person is reading or working at a computer. An untreated patient might compensate by moving the viewed material farther away than would be their previous practice.

Use of multifocul eyeglasses is the most common treatment for presbyopia. Multifocul eyeglasses have a first refractive correction for distance viewing and a second refractive correction for viewing near objects. In another proposed treatment, an ocular device that incorporates pin-hole imaging is implanted in the cornea. A pin-hole imager has a small aperture through which light is transmitted to the retina. The pin-hole imager has the effect of increasing the range of distance from the eye over which objects are in focus, referred to as the depth of focus. Increase in the depth of focus reduces the eye's need for the normal processes of accommodation, including muscle-induced lens shape change.

The cornea typically includes five major layers with the stroma as the thickest and centrally located layer. The stroma is made of overlying and interlocking collagen fibrils to form layer-like structures. The cornea ideally is transparent and colorless for optimum clarity of vision. One practice for implanting a therapeutic ocular device is to make an incision laterally displaced from the center of the cornea, e.g., outside the pupil region. An opening is formed at a layer of the cornea, frequently in the stroma into which the ocular implant can be inserted. It is desirable to limit the location of the incision to outside the pupil region to limit the deleterious effect of scarring on the patient's vision.

Some patient's vision changes to the extent that it is desirable to remove or explant a therapeutic inlay. If a relatively thin ocular device is implanted within the cornea, locating the device during removal can be difficult. This is particularly true if incisions are to be limited to the region outside the pupil region. Thus, it can prove difficult to successfully access an ocular device implanted in the cornea without further harming the patient's vision.

### SUMMARY OF THE INVENTION

There is a need for a system and device to provide reliable access to an implanted ocular device post-implantation to facilitate removal of the device. There is a need for a system and device to accurately locate the level or depth within an ocular structure where an implanted device is located without undue trauma to the patient. The system and device should avoid making incisions in the pupil region, which is a region within a boundary coincident with the largest pupil size under the normal range of conditions. It would also be advantageous for the system or device for locating an implantable ocular device not be visible to or otherwise be unobtrusive to the patient and to others interacting with the patient.

These needs are satisfied by the invention which, in one embodiment comprises an implantable ocular device comprising a therapeutic inlay and a locator structure. The therapeutic inlay is configured to be implanted at a selected region of a patient's eye at least partially within a pupil region of the eye to provide therapy for at least one vision deficiency. The locator structure is configured to be implanted within the selected region and to extend at least partially outside of the pupil region of the eye. The locator structure is accessible post-implant from outside the pupil region.

Another embodiment comprises a method of providing vision therapy. A first therapeutic inlay is implanted in a patient's eye at a selected level. The first inlay has a therapy region configured to provide therapy for at least one vision deficiency. A first locator structure is implanted at the selected level. The selected level can be identified at a later time by accessing at least a portion of the first locator structure at a location of the patient's eye outside of the therapy region.

Another embodiment comprises an inlay for implantation in a cornea of a patient between a first corneal layer and a second corneal layer, the cornea having a pupil region. The inlay comprises a mask body having an outer periphery that substantially surrounds a transmissive central portion. The mask body has an anterior surface configured to reside adjacent the first corneal layer and a posterior surface configured to reside adjacent the second corneal layer. The mask body is configured such that at least the transmissive central portion can be located in the pupil region of the cornea when the inlay is applied. The inlay also has a depth marker configured to be positioned between the first and second corneal layers outside the pupil region. The depth marker is configured to indicate the location of the mask body.

These and other objects and advantages of the invention will become more apparent from the following description taken in conjunction with the accompanying drawings.

### BRIEF DESCRIPTION OF THE DRAWINGS

FIG. 1 is a plan view of the human eye.

FIG. 2 is a cross-sectional side view of the human eye.

FIG. 3 is a cross-sectional side view of the human eye of a presbyopic patient wherein the light rays converge at a point behind the retina of the eye.

FIG. 4 is a cross-sectional side view of a presbyopic eye implanted with one embodiment of a mask wherein the light rays converge at a point on the retina.

FIG. 5 is a plan view of the human eye with a mask applied thereto.

FIG. 6 is a perspective view of one embodiment of a mask.

FIG. 7 is a frontal plan view of an embodiment of a mask with a hexagon-shaped pinhole like aperture.

FIG. 8 is a frontal plan view of an embodiment of a mask with an octagon-shaped pinhole like aperture.

FIG. 9 is a frontal plan view of an embodiment of a mask with an oval-shaped pinhole like aperture.

FIG. 10 is a frontal plan view of an embodiment of a mask with a pointed oval-shaped pinhole like aperture.

FIG. 11 is a frontal plan view of an embodiment of a mask with a star-shaped pinhole like aperture.

FIG. 12 is a frontal plan view of an embodiment of a mask with a teardrop-shaped pinhole like aperture spaced above the true center of the mask.

FIG. 13 is a frontal plan view of an embodiment of a mask with a teardrop-shaped pinhole like aperture centered within the mask.

FIG. 14 is a frontal plan view of an embodiment of a mask with a teardrop-shaped pinhole like aperture spaced below the true center of the mask.

FIG. 15 is a frontal plan view of an embodiment of a mask with a square-shaped pinhole like aperture.

FIG. 16 is a frontal plan view of an embodiment of a mask with a kidney-shaped oval pinhole like aperture.

FIG. 17 is a side view of an embodiment of a mask having varying thickness.

FIG. 18 is a side view of another embodiment of a mask having varying thickness.

FIG. 19 is a side view of an embodiment of a mask with a gel to provide opacity to the lens.

FIG. 20 is frontal plan view of an embodiment of a mask with a weave of polymeric fibers.

FIG. 21 is a side view of the mask of FIG. 20.

FIG. 22 is a frontal plan view of an embodiment of a mask having regions of varying opacity.

FIG. 23 is a side view of the mask of FIG. 22.

FIG. 24 is a frontal plan view of an embodiment of a mask that includes a centrally located pinhole like aperture and radially extending slots emanating from the center to the periphery of the mask.

FIG. 25 is a side view of the mask of FIG. 24.

FIG. 26 is a frontal plan view of an embodiment of a mask that includes a central pinhole like aperture, surrounded by a plurality of holes radially spaced from the pinhole like aperture and slots extending radially spaced from the holes and extending to the periphery of the mask.

FIG. 27 is a side view of the mask of FIG. 26.

FIG. 28 is a frontal plan view of an embodiment of a mask that includes a central pinhole like aperture, a region that includes a plurality of holes radially spaced from the aperture, and a region that includes rectangular slots spaced radially from the holes.

FIG. 29 is a side view of the mask of FIG. 28.

FIG. 30 is a frontal plan view of an embodiment of a mask that includes a non-circular pinhole like aperture, a first set of slots radially spaced from the aperture, and a region that includes a second set of slots extending to the periphery of the mask and radially spaced from the first set of slots.

FIG. 31 is a side view of the mask of FIG. 30.

FIG. 32 is a frontal plan view of an embodiment of a mask that includes a central pinhole like aperture and a plurality of holes radially spaced from the aperture.

FIG. 33 is a side view of the mask of FIG. 32.

FIG. 34 is an embodiment of a mask that includes two semi-circular mask portions.

FIG. 35 is an embodiment of a mask including two half-moon shaped portions.

FIG. 36 is an embodiment of a mask that includes a half-moon shaped region and a centrally-located pinhole like aperture.

FIG. 37 is an enlarged, diagrammatic view of an embodiment of a mask that includes particulate structure adapted for selectively controlling light transmission through the mask in a low light environment.

FIG. 38 is a view of the mask of FIG. 37 in a bright light environment.

FIG. 39 is an embodiment of a mask that includes a barcode formed on the annular region of the mask.

FIG. 40 is another embodiment of a mask that includes connectors for securing the mask within the eye.

FIG. 41 is a plan view of an embodiment of a mask made of a spiraled fibrous strand.

FIG. 42 is a plan view of the mask of FIG. 41 being removed from the eye.

FIG. 43 is a top view of another embodiment of a mask configured to increase depth of focus.

FIG. 43A is an enlarged view of a portion of the view of FIG. 43.

FIG. 44A is a cross-sectional view of the mask of FIG. 43A taken along the section plane 44-44.

FIG. 44B is a cross-sectional view similar to FIG. 44A of another embodiment of a mask.

FIG. 44C is a cross-sectional view similar to FIG. 44A of another embodiment of a mask.

FIG. 45A is a graphical representation of one arrangement of holes of a plurality of holes that may be formed on the mask of FIG. 43.

FIG. 45B is a graphical representation of another arrangement of holes of a plurality of holes that may be formed on the mask of FIG. 43.

FIG. 45C is a graphical representation of another arrangement of holes of a plurality of holes that may be formed on the mask of FIG. 43.

FIG. 46A is an enlarged view similar to that of FIG. 43A showing a variation of a mask having non-uniform size.

FIG. 46B is an enlarged view similar to that of FIG. 43A showing a variation of a mask having a non-uniform facet orientation.

FIG. 47 is a top view of another embodiment of a mask having a hole region and a peripheral region.

FIG. 48 is a flow chart illustrating one method of aligning a mask with an axis of the eye based on observation of an anatomical feature of the eye.

FIG. 49 is a flow chart illustrating one method of screening a patient for the use of a mask.

FIGS. 50A-50C show a mask, similar to those described herein, inserted beneath an epithelium sheet of a cornea.

FIGS. 51A-51C show a mask, similar to those described herein, inserted beneath a Bowman's membrane of a cornea.

FIG. 52 is a cross-sectional view of an eye illustrating a treatment of a patient wherein a flap is opened to place an implant and a location is marked for placement of the implant.

FIG. 52A is a partial plan view of the eye of FIG. 52 wherein an implant has been applied to a corneal flap and positioned with respect to a ring.

FIG. 53 is a cross-sectional view of an eye illustrating a treatment of a patient wherein a pocket is created to place an implant and a location is marked for placement of the implant.

FIG. 53A is a partial plan view of the eye of FIG. 53 wherein an implant has been positioned in a pocket and positioned with respect to a ring.

FIG. 54 is a partial side section view of an anterior portion of an eye with one embodiment of a therapeutic inlay implanted therein.

FIG. 55 is a schematic view of an anterior portion of an eye in which one embodiment of an implantable ocular device with a locator structure has been implanted.

FIG. 56 illustrates another embodiment of an implantable ocular device with a locator and a retrieval structure.

FIG. 57 illustrates one embodiment of an implantable ocular device with a locator structure that is not connected to a therapeutic inlay.

FIG. 58 illustrates a cross sectional view of the embodiment of the ocular inlay device of FIG. 4 displayed within a cornea of an eye.

FIG. 59 illustrates another embodiment of an implantable ocular device with a locator structure.

### DETAILED DESCRIPTION OF THE PREFERRED EMBODIMENT

This application is directed to ocular devices and, more particularly, to corneal inlays and other ocular implant devices with a locator structure that indicates the location of the implanted ocular device and/or facilities removal of the ocular device from the eye. One ocular implant for which these arrangements are particularly well suited includes an inlay or mask that employs pin-hole vision correction, although some of the other devices and applications in which a locator structure could be used are discussed below. The ocular devices may be applied to the eye in any manner and in any location and can be used alone or in combination with other ocular devices, e.g., with contact lenses or intraocular lenses.

I. Overview of Pin-Hole Vision Correction

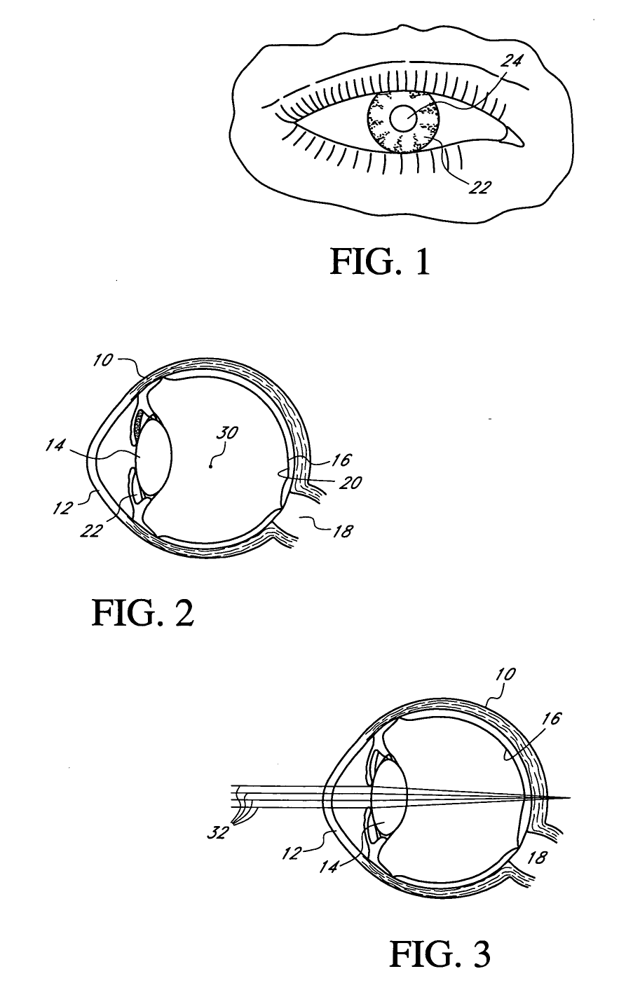

As discussed above, mask that has a pinhole aperture may be used to improve the depth of focus of a human eye. As discussed above, presbyopia is a problem of the human eye that commonly occurs in older human adults wherein the ability to focus becomes limited to inadequate range. FIGS. 1-6 illustrate how presbyopia interferes with the normal function of the eye and how a mask with a pinhole aperture mitigates the problem.

FIG. 1 shows the human eye, and FIG. 2 is a side view of the eye 10. The eye 10 includes a cornea 12 and an intraocular lens 14 posterior to the cornea 12. The cornea 12 is a first focusing element of the eye 10. The intraocular lens 14 is a second focusing element of the eye 10. The eye 10 also includes a retina 16, which lines the interior of the rear surface of the eye 10. The retina 16 includes the receptor cells which are primarily responsible for the sense of vision. The retina 16 includes a highly sensitive region, known as the macula, where signals are received and transmitted to the visual centers of the brain via the optic nerve 18. The retina 16 also includes a point with particularly high sensitivity 20, known as the fovea. As discussed in more detail in connection with FIG. 8, the fovea 20 is slightly offset from the axis of symmetry of the eye 10.

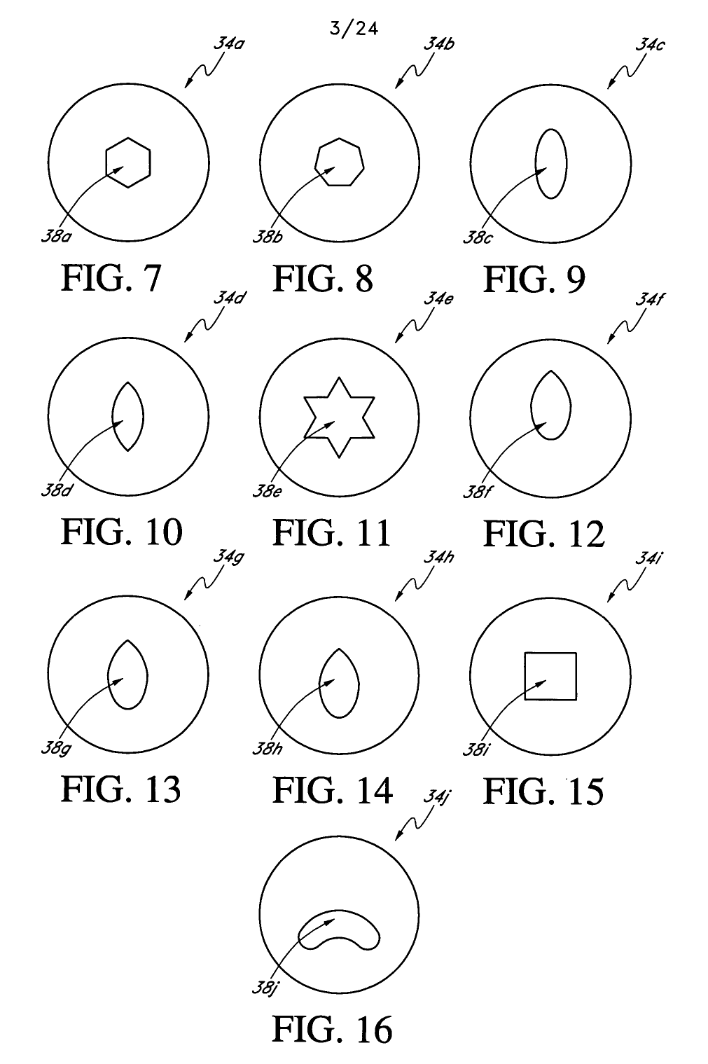

The eye 10 also includes a ring of pigmented tissue known as the iris 22. The iris 22 includes smooth muscle for controlling and regulating the size of an opening 24 in the iris 22, which is known as the pupil. An entrance pupil 26 is seen as the image of the iris 22 viewed through the cornea 12 (See FIG. 7). A central point of the entrance pupil 28 is illustrated in FIG. 7 and will be discussed further below.

The eye 10 resides in an eye-socket in the skull and is able to rotate therein about a center of rotation 30.

FIG. 3 shows the transmission of light through the eye 10 of a presbyotic patient. Due to either an aberration in the cornea 12 or the intraocular lens 14, or loss of muscle control, light rays 32 entering the eye 10 and passing through the cornea 12 and the intraocular lens 14 are refracted in such a way that the light rays 32 do not converge at a single focal point on the retina 16. FIG. 3 illustrates that in a presbyotic patient, the light rays 32 often converge at a point behind the retina 16. As a result, the patient experiences blurred vision.

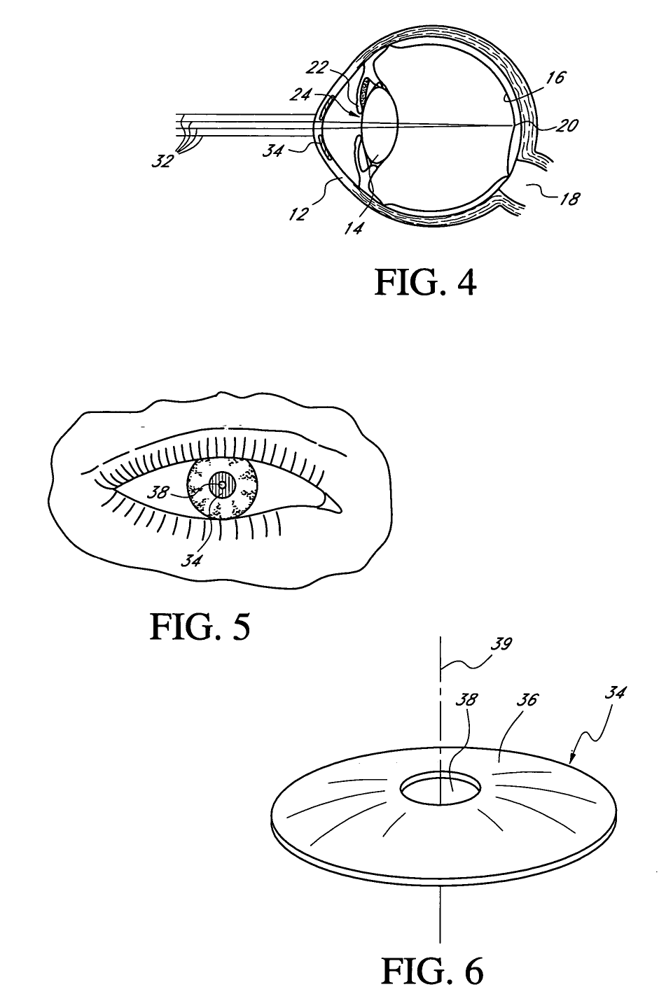

Turning now to FIG. 4, there is shown the light transmission through the eye 10 to which a mask 34 has been applied. The mask 34 is shown implanted in the cornea 12 in FIG. 4. However, as discussed below, it will be understood that the mask 34 can be, in various modes of application, implanted in the cornea 12 (as shown), used as a contact lens placed over the cornea 12, incorporated in the intraocular lens 14 (including the patient's original lens or an implanted lens), or otherwise positioned on or in the eye 10. In the illustrated embodiment, the light rays 32 that pass through the mask 34, the cornea 12, and the lens 14 converge at a single focal point on the retina 16. The light rays 32 that would not converge at the single point on retina 16 are blocked by the mask 34. As discussed below, it is desirable to position the mask 34 on the eye 10 so that the light rays 32 that pass through the mask 34 converge at the fovea 20.

Turning now to FIG. 6, there is shown one embodiment of the mask 34. As seen, the mask 34 preferably includes an annular region 36 surrounding a pinhole opening or aperture 38 substantially centrally located on the mask 34. The pinhole aperture 38 is generally located around a central axis 39, referred to herein as the optical axis of the mask 34. The pinhole aperture 38 preferably is in the shape of a circle. It has been reported that a circular aperture, such as the aperture 38 may, in some patients, produce a so-called “halo effect” where the patient perceives a shimmering image around the object being viewed. Accordingly, it may be desirable to provide an aperture 38 in a shape that diminishes, reduces, or completely eliminates the so-called “halo effect.”

II. Masks Employing Pin-Hole Correction

FIGS. 7-42 illustrate a variety of embodiments of masks that can improve the vision of a patient with presbyopia. The masks described in connection with FIG. 7-42 are similar to the mask 34, except as set forth below. Accordingly, the masks described in connection with FIGS. 7-42 can be used and applied to the eye 10 of a patient in a similar fashion to the mask 34. For example, FIG. 7 shows an embodiment of a mask 34a that includes an aperture 38a formed in the shape of a hexagon. FIG. 8 shows another embodiment of a mask 34b that includes an aperture 38b formed in the shape of an octagon. FIG. 9 shows another embodiment of a mask 34c that includes an aperture 38c formed in the shape of an oval, while FIG. 10 shows another embodiment of a mask 34d that includes an aperture 38d formed in the shape of a pointed oval. FIG. 11 shows another embodiment of a mask 34e wherein the aperture 38e is formed in the shape of a star or starburst.

FIGS. 12-14 illustrate further embodiments that have tear-drop shaped apertures. FIG. 12 shows a mask 34f that has a tear-drop shaped aperture 38f that is located above the true center of the mask 34f. FIG. 13 shows a mask 34g that has a tear-drop shaped aperture 38g that is substantially centered in the mask 34g. FIG. 14 shows a mask 34h that has a tear-drop shaped aperture 38h that is below the true center of the mask 34h. FIG. 12-14 illustrate that the position of aperture can be tailored, e.g., centered or off-center, to provide different effects. For example, an aperture that is located below the true center of a mask generally will allow more light to enter the eye because the upper portion of the aperture 34 will not be covered by the eyelid of the patient. Conversely, where the aperture is located above the true center of the mask, the aperture may be partially covered by the eyelid. Thus, the above-center aperture may permit less light to enter the eye.

FIG. 15 shows an embodiment of a mask 34i that includes an aperture 38i formed in the shape of a square. FIG. 16 shows an embodiment of a mask 34j that has a kidney-shaped aperture 38j. It will be appreciated that the apertures shown in FIGS. 7-16 are merely exemplary of non-circular apertures. Other shapes and arrangements may also be provided and are within the scope of the present invention.

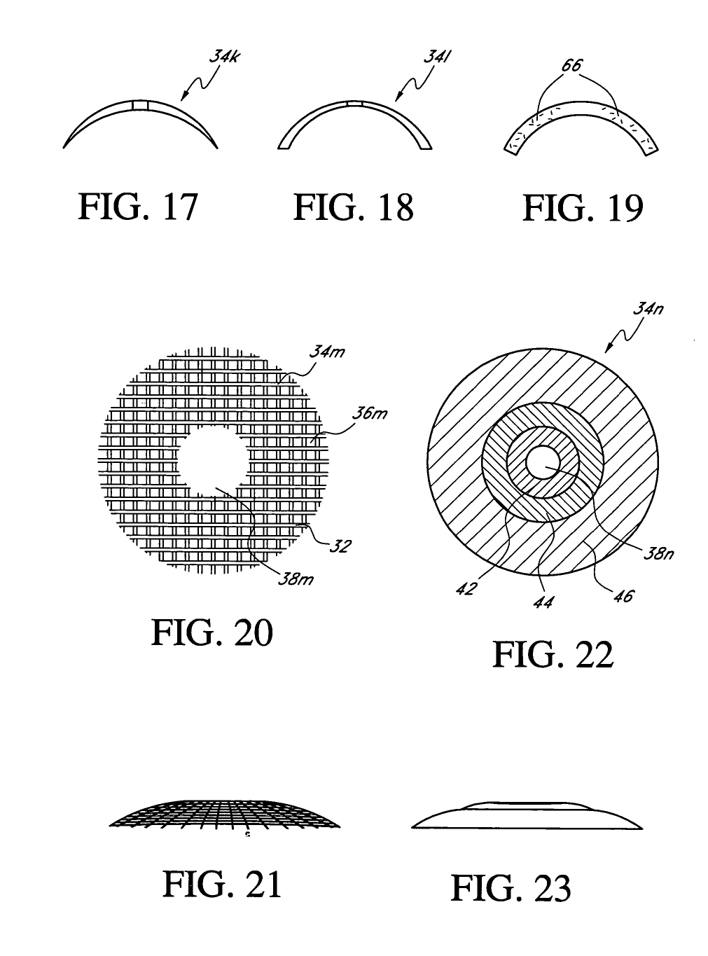

The mask 34 preferably has a constant thickness, as discussed below. However, in some embodiments, the thickness of the mask may vary between the inner periphery (near the aperture 38) and the outer periphery. FIG. 17 shows a mask 34k that has a convex profile, i.e., that has a gradually decreasing thickness from the inner periphery to the outer periphery. FIG. 18 shows a mask 341 that has a concave profile, i.e., that has a gradually increasing thickness from the inner periphery to the outer periphery. Other cross-sectional profiles are also possible.

The annular region 36 is at least partially and preferably completely opaque. The opacity of the annular region 36 prevents light from being transmitted through the mask 32 (as generally shown in FIG. 4). Opacity of the annular region 36 may be achieved in any of several different ways.

For example, in one embodiment, the material used to make mask 34 may be naturally opaque. Alternatively, the material used to make the mask 34 may be substantially clear, but treated with a dye or other pigmentation agent to render region 36 substantially or completely opaque. In still another example, the surface of the mask 34 may be treated physically or chemically (such as by etching) to alter the refractive and transmissive properties of the mask 34 and make it less transmissive to light.

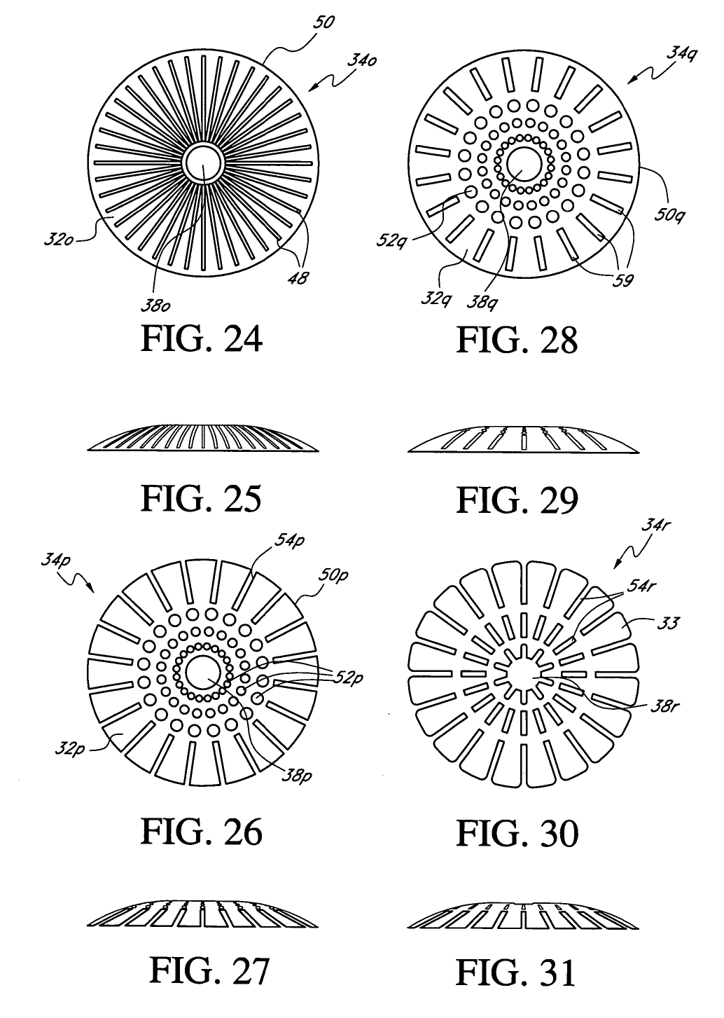

In still another alternative, the surface of the mask 34 may be treated with a particulate deposited thereon. For example, the surface of the mask 34 may be deposited with particulate of titanium, gold or carbon to provide opacity to the surface of the mask 34. In another alternative, the particulate may be encapsulated within the interior of the mask 34, as generally shown in FIG. 19. Finally, the mask 34 may be patterned to provide areas of varying light transmissivity, as generally shown in FIGS. 24-33, which are discussed in detail below.

Turning to FIG. 20, there is shown a mask 34m formed or made of a woven fabric, such as a mesh of polyester fibers. The mesh may be a cross-hatched mesh of fibers. The mask 34m includes an annular region 36m surrounding an aperture 38m. The annular region 36m comprises a plurality of generally regularly positioned apertures 36m in the woven fabric allow some light to pass through the mask 34m. The amount of light transmitted can be varied and controlled by, for example, moving the fibers closer together or farther apart, as desired. Fibers more densely distributed allow less light to pass through the annular region 36m. Alternatively, the thickness of fibers can be varied to allow more or less light through the openings of the mesh. Making the fiber strands larger results in the openings being smaller.

FIG. 22 shows an embodiment of a mask 34n that includes an annular region 36n that has sub-regions with different opacities. The opacity of the annular region 36n may gradually and progressively increase or decrease, as desired. FIG. 22 shows one embodiment where a first area 42 closest to an aperture 38n has an opacity of approximately 43%. In this embodiment, a second area 44, which is outlying with respect to the first area 42, has a greater opacity, such as 70%. In this embodiment, a third area 46, which is outlying with respect to the second area 42, has an opacity of between 85 to 100%. The graduated opacity of the type described above and shown in FIG. 22 is achieved in one embodiment by, for example, providing different degrees of pigmentation to the areas 42, 44 and 46 of the mask 34n. In another embodiment, light blocking materials of the type described above in variable degrees may be selectively deposited on the surface of a mask to achieve a graduated opacity.

In another embodiment, the mask may be formed from co-extruded rods made of material having different light transmissive properties. The co-extruded rod may then be sliced to provide disks for a plurality of masks, such as those described herein.

FIGS. 24-33 shows examples of masks that have been modified to provide regions of differing opacity. For example, FIG. 24 shows a mask 34o that includes an aperture 38o and a plurality of cutouts 48 in the pattern of radial spokes extending from near the aperture 38o to an outer periphery 50 of the mask 34o. FIG. 24 shows that the cutouts 48 are much more densely distributed about a circumference of the mask near aperture 38o than are the cutouts 48 about a circumference of the mask near the outer periphery 50. Accordingly, more light passes through the mask 34o nearer aperture 38o than near the periphery 50. The change in light transmission through the mask 34o is gradual.

FIGS. 26-27 show another embodiment of a mask 34p. The mask 34p includes an aperture 38p and a plurality of circular cutouts 49p, and a plurality of cutouts 51p. The circular cutouts 49p are located proximate the aperture 38p. The cutouts 51p are located between the circular cutouts 49p and the periphery 50p. The density of the circular cutouts 49p generally decreases from the near the aperture 38p toward the periphery 50p. The periphery 50p of the mask 34p is scalloped by the presence of the cutouts 51, which extend inward from the periphery 50p, to allow some light to pass through the mask at the periphery 50p.

FIGS. 28-29 shows another embodiment similar to that of FIGS. 26-27 wherein a mask 34q includes a plurality of circular cutouts 49q and a plurality of cutouts 51q. The cutouts 51q are disposed along the outside periphery 50q of the mask 34q, but not so as to provide a scalloped periphery.

FIGS. 30 and 31 illustrate an embodiment of a mask 34r that includes an annular region 36r that is patterned and an aperture 38r that is non-circular. As shown in FIG. 30, the aperture 38r is in the shape of a starburst. Surrounding the aperture 38r is a series of cutouts 51r that are more densely spaced toward the aperture 38r. The mask 34r includes an outer periphery 50r that is scalloped to provide additional light transmission at the outer periphery 50r.

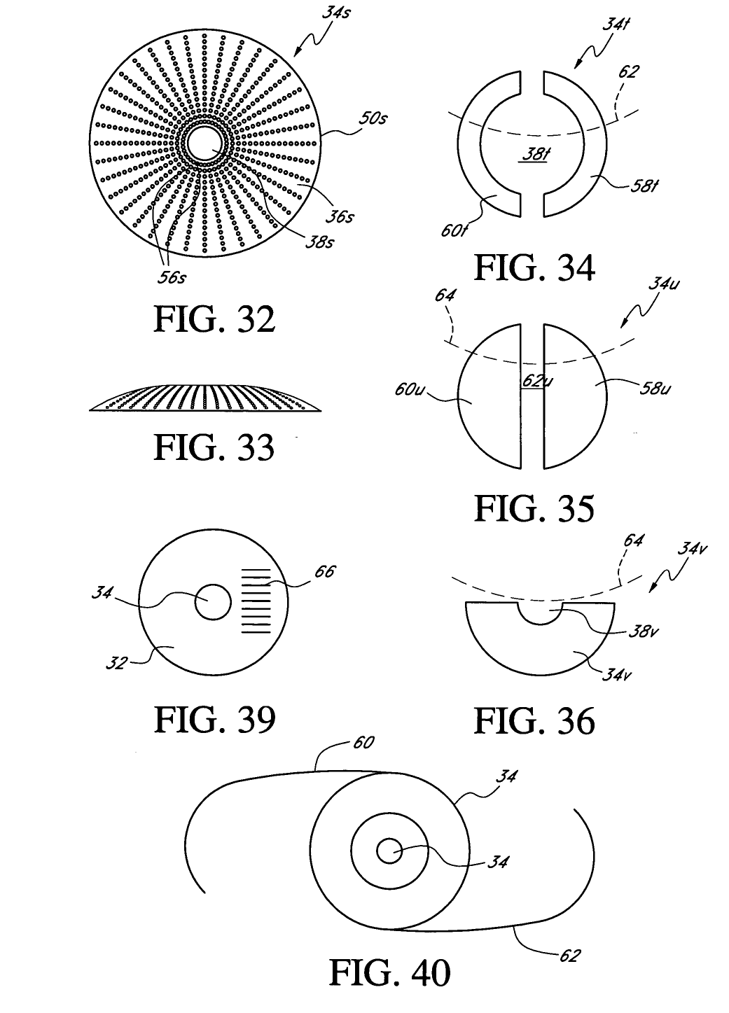

FIGS. 32 and 33 show another embodiment of a mask 34s that includes an annular region 36s and an aperture 38s. The annular region 36s is located between an outer periphery 50s of the mask 34s and the aperture 38s. The annular region 36s is patterned. In particular, a plurality of circular openings 56s is distributed over the annular region 36s of the mask 34s. It will be appreciated that the density of the openings 56s is greater near the aperture 38s than near the periphery 50s of the mask 34s. As with the examples described above, this results in a gradual increase in the opacity of the mask 34s from aperture 38s to periphery 50s.

FIGS. 34-36 show further embodiments. In particular, FIG. 34 shows a mask 34t that includes a first mask portion 58t and a second mask portion 43t. The mask portions 58t, 43t are generally “C-shaped.” As shown in FIG. 34, the mask portions 58t, 43t are implanted or inserted such that the mask portions 58t, 43t define a pinhole or aperture 38t.

FIG. 35 shows another embodiment wherein a mask 34u includes two mask portions 58u, 43u. Each mask portion 58u, 43u is in the shape of a half-moon and is configured to be implanted or inserted in such a way that the two halves define a central gap or opening 45u, which permits light to pass therethrough. Although opening 45u is not a circular pinhole, the mask portions 58u, 43u in combination with the eyelid (shown as dashed line 47) of the patient provide a comparable pinhole effect.

FIG. 36 shows another embodiment of a mask 34v that includes an aperture 38v and that is in the shape of a half-moon. As discussed in more detail below, the mask 34v may be implanted or inserted into a lower portion of the cornea 12 where, as described above, the combination of the mask 34v and the eyelid 45 provides the pinhole effect.

Other embodiments employ different ways of controlling the light transmissivity through a mask. For example, the mask may be a gel-filled disk, as shown in FIG. 19. The gel may be a hydrogel or collagen, or other suitable material that is biocompatible with the mask material and can be introduced into the interior of the mask. The gel within the mask may include particulate 53 suspended within the gel. Examples of suitable particulate are gold, titanium, and carbon particulate, which, as discussed above, may alternatively be deposited on the surface of the mask.

The material of the mask 34 may be any biocompatible polymeric material. Where a gel is used, the material is suitable for holding a gel. Examples of suitable materials for the mask 34 include the preferred polymethylmethacrylate or other suitable polymers, such as polycarbonates and the like. Of course, as indicated above, for non-gel-filled materials, a preferred material may be a fibrous material, such as a Dacron mesh.

The mask 34 may also be made to include a medicinal fluid, such as an antibiotic that can be selectively released after application, insertion, or implantation of the mask 34 into the eye of the patient. Release of an antibiotic after application, insertion, or implantation provides faster healing of the incision. The mask 34 may also be coated with other desired drugs or antibiotics. For example, it is known that cholesterol deposits can build up on the eye. Accordingly, the mask 34 may be provided with a releasable cholesterol deterring drug. The drug may be coated on the surface of the mask 34 or, in an alternative embodiment, incorporated into the polymeric material (such as PMMA) from which the mask 34 is formed.

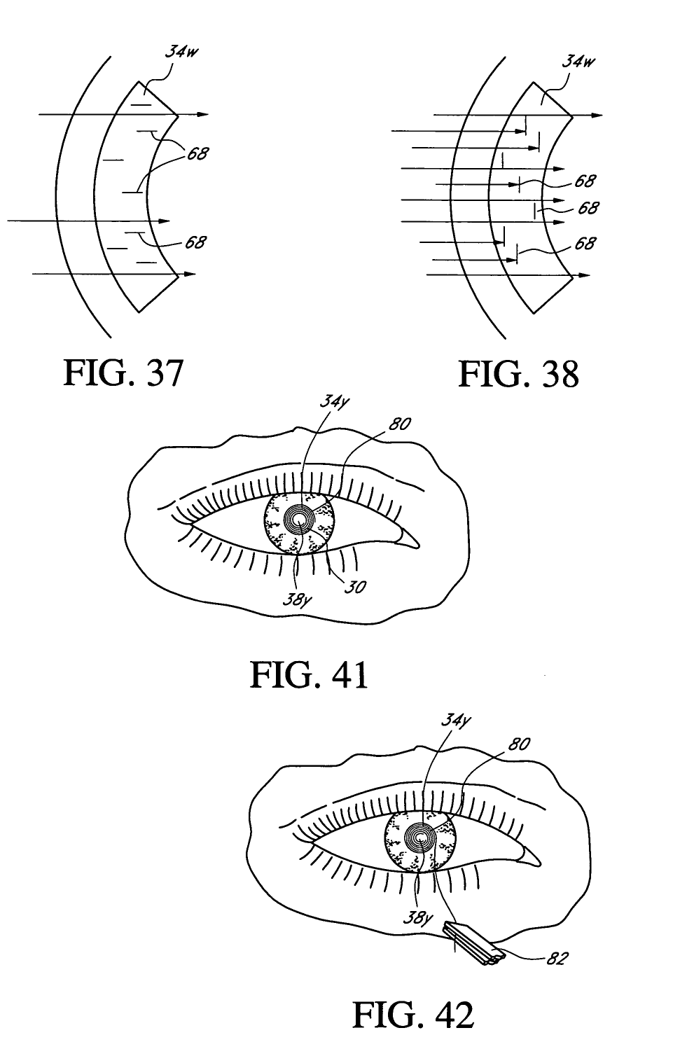

FIGS. 37 and 38 illustrate one embodiment where a mask 34w comprises a plurality of nanites 68. “Nanites” are small particulate structures that have been adapted to selectively transmit or block light entering the eye of the patient. The particles may be of a very small size typical of the particles used in nanotechnology applications. The nanites 68 are suspended in the gel or otherwise inserted into the interior of the mask 34w, as generally shown in FIGS. 37 and 38. The nanites 68 can be preprogrammed to respond to different light environments.

Thus, as shown in FIG. 37, in a high light environment, the nanites 68 turn and position themselves to substantially and selectively block some of the light from entering the eye. However, in a low light environment where it is desirable for more light to enter the eye, nanites may respond by turning or be otherwise positioned to allow more light to enter the eye, as shown in FIG. 38.

Nano-devices or nanites are crystalline structures grown in laboratories. The nanites may be treated such that they are receptive to different stimuli such as light. In accordance with one aspect of the present invention, the nanites can be imparted with energy where, in response to a low light and high light environments, they rotate in the manner described above and generally shown in FIG. 38.

Nanoscale devices and systems and their fabrication are described in Smith et al., “Nanofabrication,” Physics Today, February 1990, pp. 24-30 and in Craighead, “Nanoelectromechanical Systems,” Science, Nov. 24, 2000, Vol. 290, pp. 1502-1505, both of which are incorporated by reference herein in their entirety. Tailoring the properties of small-sized particles for optical applications is disclosed in Chen et al. “Diffractive Phase Elements Based on Two-Dimensional Artificial Dielectrics,” Optics Letters, Jan. 15, 1995, Vol. 20, No. 2, pp. 121-123, also incorporated by reference herein in its entirety.

Masks 34 made in accordance with the present invention may be further modified to include other properties. FIG. 39 shows one embodiment of a mask 34x that includes a bar code 70 or other printed indicia.

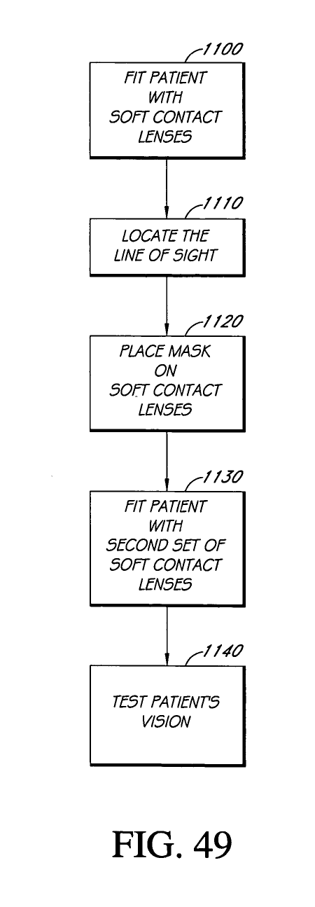

The masks described herein may be incorporated into the eye of a patient in different ways. For example, as discussed in more detail below in connection with FIG. 49, the mask 34 may be provided as a contact lens placed on the surface of the eyeball 10. Alternatively, the mask 34 may be incorporated in an artificial intraocular lens designed to replace the original lens 14 of the patient. Preferably, however, the mask 34 is provided as a corneal implant or inlay, where it is physically inserted between the layers of the cornea 12.

When used as a corneal implant, layers of the cornea 12 are peeled away to allow insertion of the mask 34. Typically, the optical surgeon (using a laser) cuts away and peels away a flap of the overlying corneal epithelium. The mask 34 is then inserted and the flap is placed back in its original position where, over time, it grows back and seals the eyeball. In some embodiments, the mask 34 is attached or fixed to the eye 10 by support strands 72 and 74 shown in FIG. 40 and generally described in U.S. Pat. No. 4,976,732, incorporated by reference herein in its entirety.

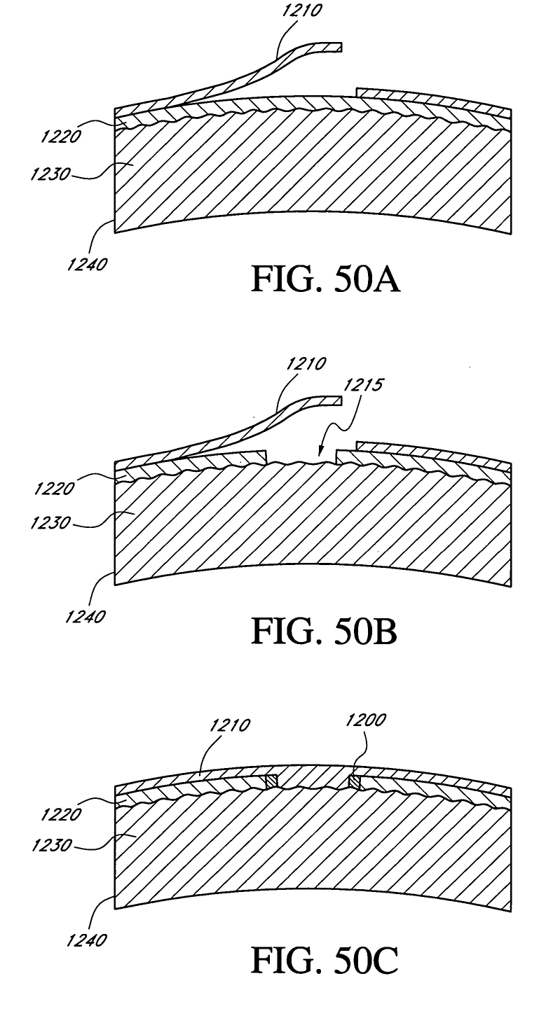

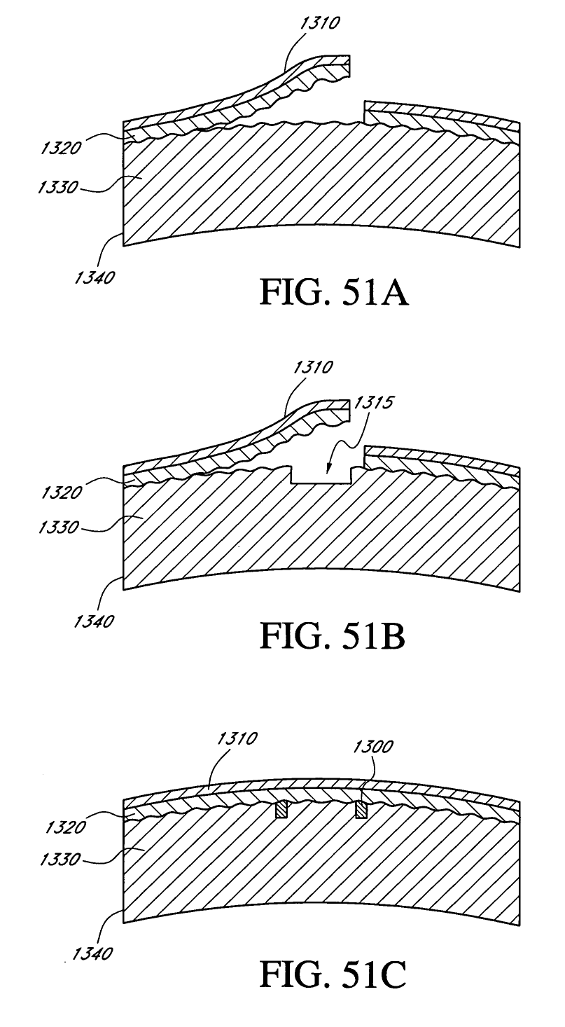

In certain circumstances, to accommodate the mask 34, the surgeon may be required to remove additional corneal tissue. Thus, in one embodiment, the surgeon may use a laser to peel away additional layers of the cornea 12 to provide a pocket that will accommodate the mask 34. Application of the mask 34 to the cornea 12 of the eye 10 of a patient is described in greater detail in connection with FIGS. 50A-51C.

Removal of the mask 34 may be achieved by simply making an additional incision in the cornea 12, lifting the flap and removing the mask 34. Alternatively, ablation techniques may be used to completely remove the mask 34.

FIGS. 41 and 42 illustrate another embodiment, of a mask 34y that includes a coiled strand 80 of a fibrous or other material. Strand 80 is coiled over itself to form the mask 34y, which may therefore be described as a spiral-like mask. This arrangement provides a pinhole or aperture 38y substantially in the center of the mask 34y. The mask 34y can be removed by a technician or surgeon who grasps the strand 80 with tweezers 82 through an opening made in a flap of the corneal 12. FIG. 42 shows this removal technique.

Further mask details are disclosed in U.S. Pat. No. 4,976,732, issued Dec. 11, 1990 and in U.S. Provisional Application Serial No. 43/473,824, filed May 28, 2003, both of which are incorporated by reference herein in their entirety.

III. Masks Configured to Reduce the Visibility of Diffraction Patterns

Many of the foregoing masks can be used to improve the depth of focus of a patient. Various additional mask embodiments are discussed below. Some of the embodiments described below include nutrient transport structures that are configured to enhance or maintain nutrient flow between adjacent tissues by facilitating transport of nutrients across the mask. The nutrient transport structures of some of the embodiments described below are configured to at least substantially prevent nutrient depletion in adjacent tissues. The nutrient transport structures can decrease negative effects due to the presence of the mask in adjacent corneal layers when the mask is implanted in the cornea, increasing the longevity of the masks. The inventors have discovered that certain arrangements of nutrient transport structures generate diffraction patterns that interfere with the vision improving effect of the masks described herein. Accordingly, certain masks are described herein that include nutrient transport structures that do not generate diffraction patterns or otherwise interfere with the vision enhancing effects of the mask embodiments.

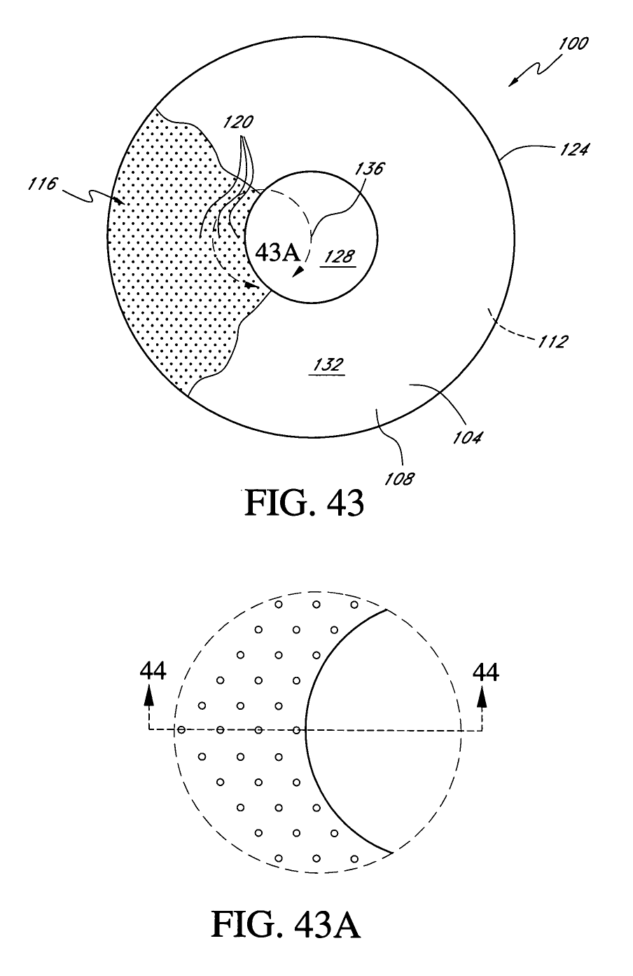

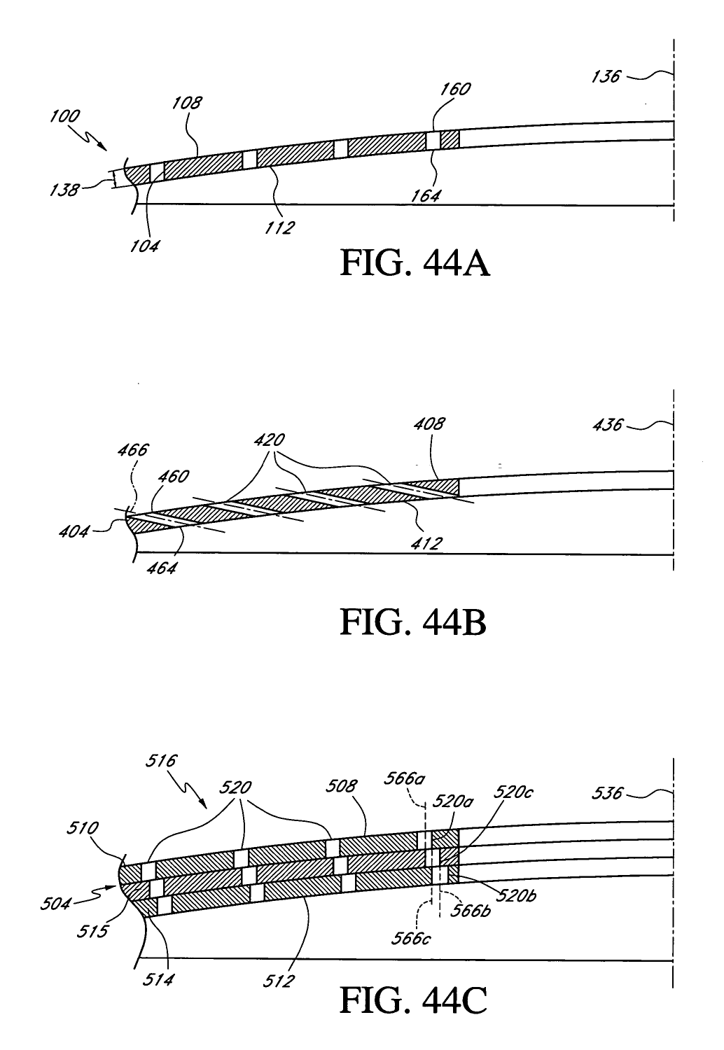

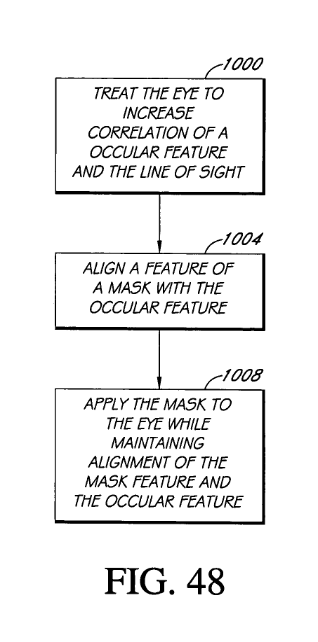

FIGS. 43-44 show one embodiment of a mask 100 configured to increase depth of focus of an eye of a patient suffering from presbyopia. The mask 100 is similar to the masks hereinbefore described, except as set forth below. Also, the mask 100 can be formed by any suitable process, such as those discussed below in connection with FIGS. 48a-48d with variations of such processes. The mask 100 is configured to be applied to an eye of a patient, e.g., by being implanted in the cornea of the patient. The mask 100 may be implanted within the cornea in any suitable manner, such as those discussed above in connection with FIGS. 50A-51C.

In one embodiment, the mask 100 includes a body 104 that has an anterior surface 108 and a posterior surface 112. In one embodiment, the body 104 is capable of substantially maintaining natural nutrient flow between the first corneal layer and the second corneal layer. In one embodiment, the material is selected to maintain at least about ninety-six percent of the natural flow of at least one nutrient (e.g., glucose) between a first corneal layer (e.g., the layer 1210) and a second corneal layer (e.g., the layer 1220). The body 104 may be formed of any suitable material, including at least one of an open cell foam material, an expanded solid material, and a substantially opaque material. In one embodiment, the material used to form the body 104 has relatively high water content.

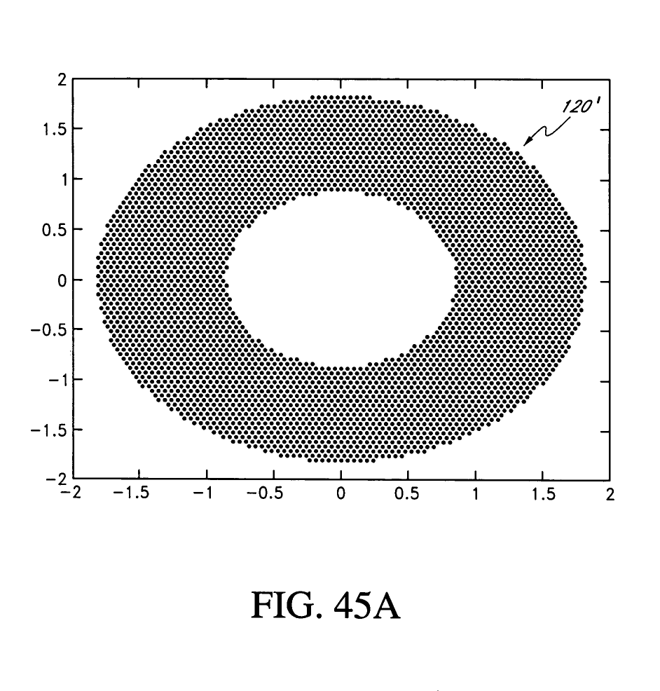

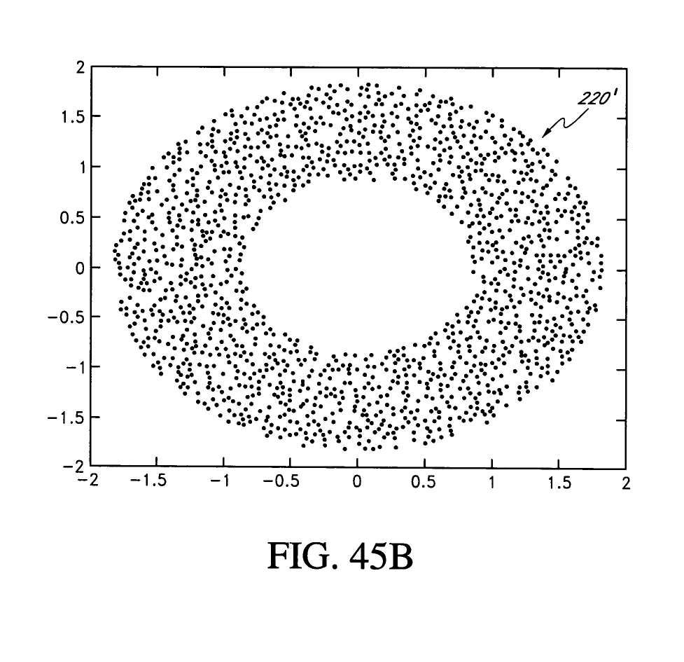

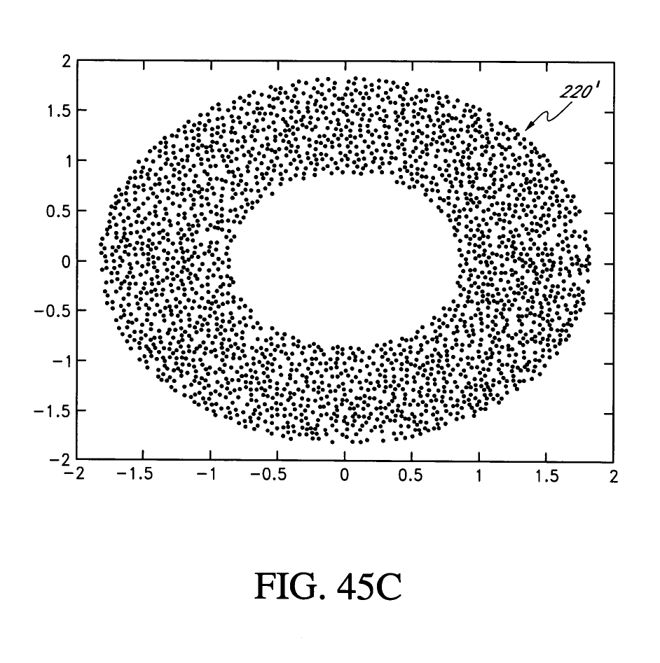

In one embodiment, the mask 100 includes and a nutrient transport structure 116. The nutrient transport structure 116 may comprise a plurality of holes 120. The holes 120 are shown on only a portion of the mask 100, but the holes 120 preferably are located throughout the body 104 in one embodiment. In one embodiment, the holes 120 are arranged in a hex pattern, which is illustrated by a plurality of locations 120′ in FIG. 45A. As discussed below, a plurality of locations may be defined and later used in the later formation of a plurality of holes 120 on the mask 100. The mask 100 has an outer periphery 124 that defines an outer edge of the body 104. In some embodiments, the mask 100 includes an aperture 128 at least partially surrounded by the outer periphery 124 and a non-transmissive portion 132 located between the outer periphery 124 and the aperture 128.

Preferably the mask 100 is symmetrical, e.g., symmetrical about a mask axis 136. In one embodiment, the outer periphery 124 of the mask 100 is circular. The masks in general have has a diameter within the range of from about 3 mm to about 8 mm, often within the range of from about 3.5 mm to about 6 mm, and less than about 6 mm in one embodiment. In another embodiment, the mask is circular and has a diameter in the range of 4 to 6 mm. In another embodiment, the mask 100 is circular and has a diameter of less than 4 mm. The outer periphery 124 has a diameter of about 3.8 mm in another embodiment. In some embodiments, masks that are asymmetrical or that are not symmetrical about a mask axis provide benefits, such as enabling a mask to be located or maintained in a selected position with respect to the anatomy of the eye.

The body 104 of the mask 100 may be configured to coupled with a particular anatomical region of the eye. The body 104 of the mask 100 may be configured to conform to the native anatomy of the region of the eye in which it is to be applied. For example, where the mask 100 is to be coupled with an ocular structure that has curvature, the body 104 may be provided with an amount of curvature along the mask axis 136 that corresponds to the anatomical curvature. For example, one environment in which the mask 100 may be deployed is within the cornea of the eye of a patient. The cornea has an amount of curvature that varies from person to person about a substantially constant mean value within an identifiable group, e.g., adults. When applying the mask 100 within the cornea, at least one of the anterior and posterior surfaces 108, 112 of the mask 100 may be provided with an amount of curvature corresponding to that of the layers of the cornea between which the mask 100 is applied.

In some embodiments, the mask 100 has a desired amount of optical power. Optical power may be provided by configuring the at least one of the anterior and posterior surfaces 108, 112 with curvature. In one embodiment, the anterior and posterior surfaces 108, 112 are provided with different amounts of curvature. In this embodiment, the mask 100 has varying thickness from the outer periphery 124 to the aperture 128.

In one embodiment, one of the anterior surface 108 and the posterior surface 112 of the body 104 is substantially planar. In one planar embodiment, very little or no uniform curvature can be measured across the planar surface. In another embodiment, both of the anterior and posterior surfaces 108, 112 are substantially planar. In general, the thickness of the inlay may be within the range of from about 1 micron to about 40 micron, and often in the range of from about 5 micron to about 20 micron. In one embodiment, the body 104 of the mask 100 has a thickness 138 of between about 5 micron and about 10 micron. In one embodiment, the thickness 138 of the mask 100 is about 5 micron. In another embodiment, the thickness 138 of the mask 100 is about 8 micron. In another embodiment, the thickness 138 of the mask 100 is about 10 micron.

Thinner masks generally are more suitable for applications wherein the mask 100 is implanted at a relatively shallow location in (e.g., close to the anterior surface of) the cornea. In thinner masks, the body 104 may be sufficiently flexible such that it can take on the curvature of the structures with which it is coupled without negatively affecting the optical performance of the mask 100. In one application, the mask 100 is configured to be implanted about 5 um beneath the anterior surface of the cornea. In another application, the mask 100 is configured to be implanted about 52 um beneath the anterior surface of the cornea. In another application, the mask 100 is configured to be implanted about 125 um beneath the anterior surface of the cornea. Further details regarding implanting the mask 100 in the cornea are discussed above in connection with FIGS. 50A-51C.

A substantially planar mask has several advantages over a non-planar mask. For example, a substantially planar mask can be fabricated more easily than one that has to be formed to a particular curvature. In particular, the process steps involved in inducing curvature in the mask 100 can be eliminated. Also, a substantially planar mask may be more amenable to use on a wider distribution of the patient population (or among different sub-groups of a broader patient population) because the substantially planar mask uses the curvature of each patient's cornea to induce the appropriate amount of curvature in the body 104.

In some embodiments, the mask 100 is configured specifically for the manner and location of coupling with the eye. In particular, the mask 100 may be larger if applied over the eye as a contact lens or may be smaller if applied within the eye posterior of the cornea, e.g., proximate a surface of the lens of the eye. As discussed above, the thickness 138 of the body 104 of the mask 100 may be varied based on where the mask 100 is implanted. For implantation at deeper levels within the cornea, a thicker mask may be advantageous. Thicker masks are advantageous in some applications. For example, they are generally easier to handle, and therefore are easier to fabricate and to implant. Thicker masks may benefit more from having a preformed curvature than thinner masks. A thicker mask could be configured to have little or no curvature prior to implantation if it is configured to conform to the curvature of the native anatomy when applied.

The aperture 128 is configured to transmit substantially all incident light along the mask axis 136. The non-transmissive portion 132 surrounds at least a portion of the aperture 128 and substantially prevents transmission of incident light thereon. As discussed in connection with the above masks, the aperture 128 may be a through-hole in the body 104 or a substantially light transmissive (e.g., transparent) portion thereof. The aperture 128 of the mask 100 generally is defined within the outer periphery 124 of the mask 100. The aperture 128 may take any of suitable configurations, such as those described above in connection with FIGS. 6-42.

In one embodiment, the aperture 128 is substantially circular and is substantially centered in the mask 100. The size of the aperture 128 may be any size that is effective to increase the depth of focus of an eye of a patient suffering from presbyopia. For example, the aperture 128 can be circular, having a diameter of less than about 2.2 mm in one embodiment. In another embodiment, the diameter of the aperture is between about 1.8 mm and about 2.2 mm. In another embodiment, the aperture 128 is circular and has a diameter of about 1.8 mm or less. Most apertures will have a diameter within the range of from about 1.0 mm to about 2.5 mm, and often within the range of from about 1.3 mm to about 1.9 mm.

The non-transmissive portion 132 is configured to prevent transmission of radiant energy through the mask 100. For example, in one embodiment, the non-transmissive portion 132 prevents transmission of substantially all of at least a portion of the spectrum of the incident radiant energy. In one embodiment, the non-transmissive portion 132 is configured to prevent transmission of substantially all visible light, e.g., radiant energy in the electromagnetic spectrum that is visible to the human eye. The non-transmissive portion 132 may substantially prevent transmission of radiant energy outside the range visible to humans in some embodiments.

As discussed above in connection with FIG. 3, preventing transmission of light through the non-transmissive portion 132 decreases the amount of light that reaches the retina and the fovea that would not converge at the retina and fovea to form a sharp image. As discussed above in connection with FIG. 4, the size of the aperture 128 is such that the light transmitted therethrough generally converges at the retina or fovea. Accordingly, a much sharper image is presented to the eye than would otherwise be the case without the mask 100.

In one embodiment, the non-transmissive portion 132 prevents transmission of about 90 percent of incident light. In another embodiment, the non-transmissive portion 132 prevents transmission of about 92 percent of all incident light. The non-transmissive portion 132 of the mask 100 may be configured to be opaque to prevent the transmission of light. As used herein the term “opaque” is intended to be a broad term meaning capable of preventing the transmission of radiant energy, e.g., light energy, and also covers structures and arrangements that absorb or otherwise block all or less than all or at least a substantial portion of the light. In one embodiment, at least a portion of the body 104 is configured to be opaque to more than 99 percent of the light incident thereon.

As discussed above, the non-transmissive portion 132 may be configured to prevent transmission of light without absorbing the incident light. For example, the mask 100 could be made reflective or could be made to interact with the light in a more complex manner, as discussed in U.S. Pat. No. 6,551,424, issued Apr. 29, 2003, which is hereby incorporated by reference herein in its entirety.

As discussed above, the mask 100 also has a nutrient transport structure that in some embodiments comprises the plurality of holes 120. The presence of the plurality of holes 120 (or other transport structure) may affect the transmission of light through the non-transmissive portion 132 by potentially allowing more light to pass through the mask 100. In one embodiment, the non-transmissive portion 132 is configured to absorb about 99 percent or more of the incident light from passing through the mask 100 without holes 120 being present. The presence of the plurality of holes 120 allows more light to pass through the non-transmissive portion 132 such that only about 92 percent of the light incident on the non-transmissive portion 132 is prevented from passing through the non-transmissive portion 132. The holes 120 may reduce the benefit of the aperture 128 on the depth of focus of the eye by allowing more light to pass through the non-transmissive portion to the retina.

Reduction in the depth of focus benefit of the aperture 128 due to the holes 120 is balanced by the nutrient transmission benefits of the holes 120. In one embodiment, the transport structure 116 (e.g., the holes 120) is capable of substantially maintaining natural nutrient flow from a first corneal layer (i.e., one that is adjacent to the anterior surface 108 of the mask 100) to the second corneal layer (i.e., one that is adjacent to the posterior surface 112 of the mask 100). The plurality of holes 120 are configured to enable nutrients to pass through the mask 100 between the anterior surface 108 and the posterior surface 112. As discussed above, the holes 120 of the mask 100 shown in FIG. 43 may be located anywhere on the mask 100. Other mask embodiments described herein below locate substantially all of the nutrient transport structure in one or more regions of a mask.

The holes 120 of FIG. 43 extends at least partially between the anterior surface 108 and the posterior surface 112 of the mask 100. In one embodiment, each of the holes 120 includes a hole entrance 140 and a hole exit 164. The hole entrance 140 is located adjacent to the anterior surface 108 of the mask 100. The hole exit 164 is located adjacent to the posterior surface 112 of the mask 100. In one embodiment, each of the holes 120 extends the entire distance between the anterior surface 108 and the posterior surface 112 of the mask 100.

The transport structure 116 is configured to maintain the transport of one or more nutrients across the mask 100. The transport structure 116 of the mask 100 provides sufficient flow of one or more nutrients across the mask 100 to prevent depletion of nutrients at least at one of the first and second corneal layers (e.g., the layers 1210 and 1220). One nutrient of particular importance to the viability of the adjacent corneal layers is glucose. The transport structure 116 of the mask 100 provides sufficient flow of glucose across the mask 100 between the first and second corneal layers to prevent glucose depletion that would harm the adjacent corneal tissue. Thus, the mask 100 is capable of substantially maintaining nutrient flow (e.g., glucose flow) between adjacent corneal layers. In one embodiment, the nutrient transport structure 116 is configured to prevent depletion of more than about 4 percent of glucose (or other biological substance) in adjacent tissue of at least one of the first corneal layer and the second corneal layer.

The holes 120 may be configured to maintain the transport of nutrients across the mask 100. In one embodiment, the holes 120 are formed with a diameter of about 0.015 mm or more. In another embodiment, the holes have a diameter of about 0.020 mm. In another embodiment, the holes have a diameter of about 0.025 mm. In another embodiment, the holes 120 have a diameter in the range of about 0.020 mm to about 0.029 mm. The number of holes in the plurality of holes 120 is selected such that the sum of the surface areas of the hole entrances 140 of all the holes 100 comprises about 5 percent or more of surface area of the anterior surface 108 of the mask 100. In another embodiment, the number of holes 120 is selected such that the sum of the surface areas of the hole exits 164 of all the holes 120 comprises about 5 percent or more of surface area of the posterior surface 112 of the mask 100. In another embodiment, the number of holes 120 is selected such that the sum of the surface areas of the hole exits 164 of all the holes 120 comprises about 5 percent or more of surface area of the posterior surface 112 of the mask 112 and the sum of the surface areas of the hole entrances 140 of all the holes 120 comprises about 5 percent or more of surface area of the anterior surface 108 of the mask 100.

Each of the holes 120 may have a relatively constant cross-sectional area. In one embodiment, the cross-sectional shape of each of the holes 120 is substantially circular. Each of the holes 120 may comprise a cylinder extending between the anterior surface 108 and the posterior surface 112.

The relative position of the holes 120 is of interest in some embodiments. As discussed above, the holes 120 of the mask 100 are hex-packed, e.g., arranged in a hex pattern. In particular, in this embodiment, each of the holes 120 is separated from the adjacent holes 120 by a substantially constant distance, sometimes referred to herein as a hole pitch. In one embodiment, the hole pitch is about 0.045 mm.

In a hex pattern, the angles between lines of symmetry are approximately 43 degrees. The spacing of holes along any line of holes is generally within the range of from about 30 microns to about 100 microns, and, in one embodiment, is approximately 43 microns. The hole diameter is generally within the range of from about 10 microns to about 100 microns, and in one embodiment, is approximately 20 microns. The hole spacing and diameter are related if you want to control the amount of light coming through. The light transmission is a function of the sum of hole areas as will be understood by those of skill in the art in view of the disclosure herein.

The embodiment of FIG. 43 advantageously enables nutrients to flow from the first corneal layer to the second corneal layer. The inventors have discovered that negative visual effects can arise due to the presence of the transport structure 116. For example, in some cases, a hex packed arrangement of the holes 120 can generate diffraction patterns visible to the patient. For example, patients might observe a plurality of spots, e.g., six spots, surrounding a central light with holes 120 having a hex patterned.

The inventors have discovered a variety of techniques that produce advantageous arrangements of a transport structure such that diffraction patterns and other deleterious visual effects do not substantially inhibit other visual benefits of a mask. In one embodiment, where diffraction effects would be observable, the nutrient transport structure is arranged to spread the diffracted light out uniformly across the image to eliminate observable spots. In another embodiment, the nutrient transport structure employs a pattern that substantially eliminates diffraction patterns or pushes the patterns to the periphery of the image.

FIG. 45B-45C show two embodiments of patterns of holes 220 that may be applied to a mask that is otherwise substantially similar to the mask 100. The holes 220 of the hole patterns of FIGS. 45A-45B are spaced from each other by a random hole spacing or hole pitch. In other embodiments discussed below, holes are spaced from each other by a non-uniform amount, e.g., not a random amount. In one embodiment, the holes 220 have a substantially uniform shape (cylindrical shafts having a substantially constant cross-sectional area). FIG. 45C illustrates a plurality of holes 220 separated by a random spacing, wherein the density of the holes is greater than that of FIG. 45B. Generally, the higher the percentage of the mask body that has holes the more the mask will transport nutrients in a manner similar to the native tissue. One way to provide a higher percentage of hole area is to increase the density of the holes. Increase hole density can also permit smaller holes to achieve the same nutrient transport as is achieved by less dense, larger holes.

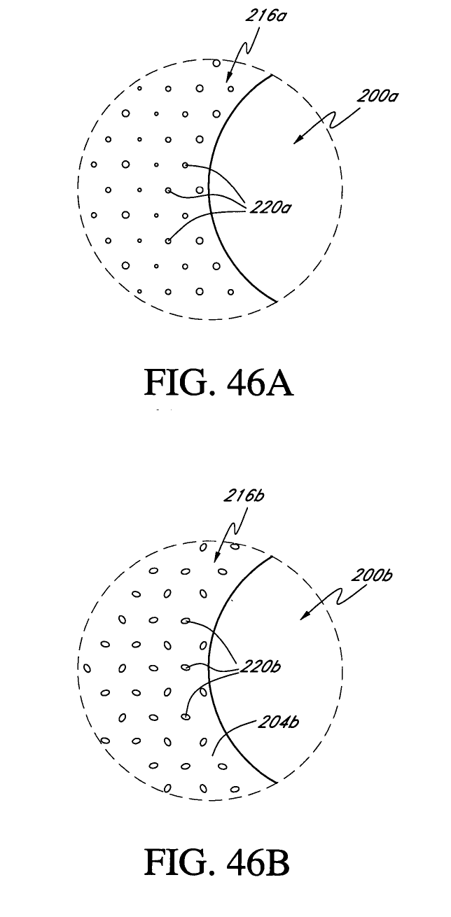

FIG. 46A shows a portion of another mask 200a that is substantially similar to the mask 100, except as set forth below. The mask 200a can be formed by any suitable process, such as those discussed below in connection with FIGS. 48a-48d and with variations of such processes. The mask 200a has a nutrient transport structure 216a that includes a plurality of holes 220a. A substantial number of the holes 220a have a non-uniform size. The holes 220a may be uniform in cross-sectional shape. The cross-sectional shape of the holes 220a is substantially circular in one embodiment. The holes 220a may be circular in shape and have the same diameter from a hole entrance to a hole exit, but are otherwise non-uniform in at least one aspect, e.g., in size. It may be preferable to vary the size of a substantial number of the holes by a random amount. In another embodiment, the holes 220a are non-uniform (e.g., random) in size and are separated by a non-uniform (e.g., a random) spacing.

FIG. 46B illustrates another embodiment of a mask 200b that is substantially similar to the mask 100, except as set forth below. Also, the mask 200b can be formed by any suitable process, such as those discussed below in connection with FIGS. 48a-48d and with variations of such processes. The mask 200b includes a body 204b. The mask 200b has a transport structure 216b that includes a plurality of holes 220b with a non-uniform facet orientation. In particular, each of the holes 220b has a hole entrance that may be located at an anterior surface of the mask 200b. A facet of the hole entrance is defined by a portion of the body 204b of the mask 200b surrounding the hole entrance. The facet is the shape of the hole entrance at the anterior surface. In one embodiment, most or all the facets have an elongate shape, e.g., an oblong shape, with a long axis and a short axis that is perpendicular to the long axis. The facets may be substantially uniform in shape. In one embodiment, the orientation of facets is not uniform. For example, a substantial number of the facets may have a non-uniform orientation. In one arrangement, a substantial number of the facets have a random orientation. In some embodiments, the facets are non-uniform (e.g., random) in shape and are non-uniform (e.g., random) in orientation.

Other embodiments may be provided that vary at least one aspect, including one or more of the foregoing aspects, of a plurality of holes to reduce the tendency of the holes to produce visible diffraction patterns or patterns that otherwise reduce the vision improvement that may be provided by a mask with an aperture, such as any of those described above. For example, in one embodiment, the hole size, shape, and orientation of at least a substantial number of the holes may be varied randomly or may be otherwise non-uniform.

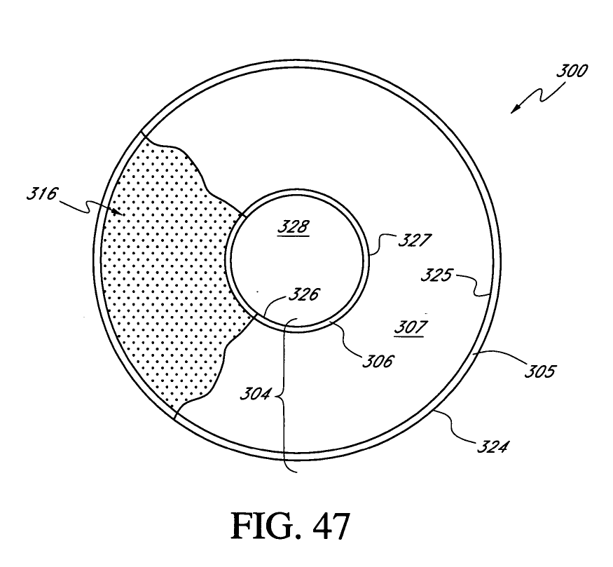

FIG. 47 shows another embodiment of a mask 300 that is substantially similar to any of the masks hereinbefore described, except as set forth below. Also, the mask 300 can be formed by any suitable process, such as those discussed below in connection with FIGS. 48a-48d and with variations of such processes. The mask 300 includes a body 304. The body 304 has an outer peripheral region 305, an inner peripheral region 306, and a hole region 307. The hole region 307 is located between the outer peripheral region 305 and the outer peripheral region 306. The body 304 may also include an aperture region, where the aperture (discussed below) is not a through hole. The mask 300 also includes a nutrient transport structure 316. In one embodiment, the nutrient transport structure includes a plurality of holes. At least a substantial portion of the holes (e.g., all of the holes) are located in the hole region 307. As above, only a portion of the nutrient structure 316 is shown for simplicity. But it should be understood that the hole may be located through the hole region 307.

The outer peripheral region 305 may extend from an outer periphery 324 of the mask 300 to a selected outer circumference 326 of the mask 300. The selected outer circumference 325 of the mask 300 is located a selected radial distance from the outer periphery 324 of the mask 300. In one embodiment, the selected outer circumference 325 of the mask 300 is located about 0.05 mm from the outer periphery 324 of the mask 300.

The inner peripheral region 306 may extend from an inner location, e.g., an inner periphery 326 adjacent an aperture 328 of the mask 300 to a selected inner circumference 327 of the mask 300. The selected inner circumference 327 of the mask 300 is located a selected radial distance from the inner periphery 326 of the mask 300. In one embodiment, the selected inner circumference 327 of the mask 300 is located about 0.05 mm from the inner periphery 326.

The mask 300 may be the product of a process that involves random selection of a plurality of locations and formation of holes on the mask 300 corresponding to the locations. As discussed further below, the method can also involve determining whether the selected locations satisfy one or more criteria. For example, one criterion prohibits all, at least a majority, or at least a substantial portion of the holes from being formed at locations that correspond to the inner or outer peripheral regions 305, 306. Another criterion prohibits all, at least a majority, or at least a substantial portion of the holes from being formed too close to each other. For example, such a criterion could be used to assure that a wall thickness, e.g., the shortest distance between adjacent holes, is not less than a predetermined amount. In one embodiment, the wall thickness is prevented from being less than about 20 microns.

In a variation of the embodiment of FIG. 47, the outer peripheral region 305 is eliminated and the hole region 307 extends from the inner peripheral region 306 to an outer periphery 324. In another variation of the embodiment of FIG. 47, the inner peripheral region 306 is eliminated and the hole region 307 extends from the outer peripheral region 305 to an inner periphery 326.

FIG. 44B shows a mask 400 that is similar to the mask 100 except as set forth below. The mask 400 can be formed by any suitable process, such as those discussed below in connection with FIGS. 48a-48d and with variations of such processes. The mask 400 includes a body 404 that has an anterior surface 408 and a posterior surface 412. The mask 400 also includes a nutrient transport structure 4316 that, in one embodiment, includes a plurality of holes 420. The holes 420 are formed in the body 404 so that nutrient transport is provided but transmission of radiant energy (e.g., light) to the retinal locations adjacent the fovea through the holes 404 is substantially prevented. In particular, the holes 404 are formed such that when the eye with which the mask 1000 is coupled is directed at an object to be viewed, light conveying the image of that object that enters the holes 420 cannot exit the holes along a path ending near the fovea.

In one embodiment, each of the holes 420 has a hole entrance 460 and a hole exit 464. Each of the holes 420 extends along a transport axis 466. The transport axis 466 is formed to substantially prevent propagation of light from the anterior surface 408 to the posterior surface 412 through the holes 420. In one embodiment, at least a substantial number of the holes 420 have a size to the transport axis 466 that is less than a thickness of the mask 400. In another embodiment, at least a substantial number of the holes 420 have a longest dimension of a perimeter at least at one of the anterior or posterior surfaces 408, 412 (e.g., a facet) that is less than a thickness of the mask 400. In some embodiments, the transport axis 466 is formed at an angle with respect to a mask axis 436 that substantially prevents propagation of light from the anterior surface 408 to the posterior surface 412 through the hole 420. In another embodiment, the transport axis 466 of one or more holes 420 is formed at an angle with respect to the mask axis 436 that is large enough to prevent the projection of most of the hole entrance 460 from overlapping the hole exit 464.

In one embodiment, the hole 420 is circular in cross-section and has a diameter between about 0.5 micron and about 8 micron and the transport axis 466 is between 5 and 85 degrees. The length of each of the holes 420 (e.g., the distance between the anterior surface 408 and the posterior surface 412) is between about 8 and about 92 micron. In another embodiment, the diameter of the holes 420 is about 5 micron and the transport angle is about 40 degrees or more. As the length of the holes 420 increases it may be desirable to include additional holes 420. In some cases, additional holes 420 counteract the tendency of longer holes to reduce the amount of nutrient flow through the mask 400.

FIG. 44C shows another embodiment of a mask 500 similar to the mask 100, except as set forth below. The mask 500 can be formed by any suitable process, such as those discussed below in connection with FIGS. 48a-48d and with variations of such processes. The mask 500 includes a body 504 that has an anterior surface 508, a first mask layer 510 adjacent the anterior surface 508, a posterior surface 512, a second mask layer 514 adjacent the posterior surface 512, and a third mask layer 515 located between the first mask layer 510 and the second mask layer 514. The mask 500 also includes a nutrient transport structure 516 that, in one embodiment, includes a plurality of holes 520. The holes 520 are formed in the body 504 so that nutrient are transported across the mask, as discussed above, but transmission of radiant energy (e.g., light) to retinal locations adjacent the fovea through the holes 504 is substantially prevented. In particular, the holes 504 are formed such that when the eye with which the mask 500 is coupled is directed at an object to be viewed, light conveying the image of that object that enters the holes 520 cannot exit the holes along a path ending near the fovea.

In one embodiment, at least one of the holes 520 extends along a non-linear path that substantially prevents propagation of light from the anterior surface to the posterior surface through the at least one hole. In one embodiment, the mask 500 includes a first hole portion 520a that extends along a first transport axis 566a, the second mask layer 514 includes a second hole portion 520b extending along a second transport axis 566b, and the third mask layer 515 includes a third hole portion 520c extending along a third transport axis 566c. The first, second, and third transport axes 566a, 566b, 566c preferably are not collinear. In one embodiment, the first and second transport axes 566a, 566b are parallel but are off-set by a first selected amount. In one embodiment, the second and third transport axes 566b, 566c are parallel but are off-set by a second selected amount. In the illustrated embodiment, each of the transport axes 566a, 566b, 566c are off-set by one-half of the width of the hole portions 520a, 520b, 520c. Thus, the inner-most edge of the hole portion 520a is spaced from the axis 536 by a distance that is equal to or greater than the distance of the outer-most edge of the hole portion 520b from the axis 536. This spacing substantially prevents light from passing through the holes 520 from the anterior surface 508 to the posterior surface 512.

In one embodiment, the first and second amounts are selected to substantially prevent the transmission of light therethrough. The first and second amounts of off-set may be achieved in any suitable fashion. One technique for forming the hole portions 520a, 520b, 520c with the desired off-set is to provide a layered structure. As discussed above, the mask 500 may include the first layer 510, the second layer 514, and the third layer 515. FIG. 44C shows that the mask 500 can be formed with three layers. In another embodiment, the mask 500 is formed of more than three layers. Providing more layers may advantageously further decrease the tendency of light to be transmitted through the holes 490 onto the retina. This has the benefit of reducing the likelihood that a patient will observe or otherwise perceive a patter that will detract from the vision benefits of the mask 500. A further benefit is that less light will pass through the mask 500, thereby enhancing the depth of focus increase due to the pin-hole sized aperture formed therein.

In any of the foregoing mask embodiments, the body of the mask may be formed of a material selected to provide adequate nutrient transport and to substantially prevent negative optic effects, such as diffraction, as discussed above. In various embodiments, the masks are formed of an open cell foam material. In another embodiment, the masks are formed of an expanded solid material.

As discussed above in connection with FIGS. 45B and 45C, various random patterns of holes may advantageously be provided for nutrient transport. In some embodiment, it may be sufficient to provide regular patterns that are non-uniform in some aspect. Non-uniform aspects to the holes may be provided by any suitable technique.

In a first step of one technique, a plurality of locations 220′ is generated. The locations 220′ are a series of coordinates that may comprise a non-uniform pattern or a regular pattern. The locations 220′ may be randomly generated or may be related by a mathematical relationship (e.g., separated by a fixed spacing or by an amount that can be mathematically defined). In one embodiment, the locations are selected to be separated by a constant pitch or spacing and may be hex packed.

In a second step, a subset of the locations among the plurality of locations 220′ is modified to maintain a performance characteristic of the mask. The performance characteristic may be any performance characteristic of the mask. For example, the performance characteristic may relate to the structural integrity of the mask. Where the plurality of locations 220′ is selected at random, the process of modifying the subset of locations may make the resulting pattern of holes in the mask a “pseudo-random” pattern.

Where a hex packed pattern of locations (such as the locations 120′ of FIG. 45A) is selected in the first step, the subset of locations may be moved with respect to their initial positions as selected in the first step. In one embodiment, each of the locations in the subset of locations is moved by an amount equal to a fraction of the hole spacing. For example, each of the locations in the subset of locations may be moved by an amount equal to one-quarter of the hole spacing. Where the subset of locations is moved by a constant amount, the locations that are moved preferably are randomly or pseudo-randomly selected. In another embodiment, the subset of location is moved by a random or a pseudo-random amount.

In one technique, an outer peripheral region is defined that extends between the outer periphery of the mask and a selected radial distance of about 0.05 mm from the outer periphery. In another embodiment, an inner peripheral region is defined that extends between an aperture of the mask and a selected radial distance of about 0.05 mm from the aperture. In another embodiment, an outer peripheral region is defined that extends between the outer periphery of the mask and a selected radial distance and an inner peripheral region is defined that extends between the aperture of the mask and a selected radial distance from the aperture. In one technique, the subset of location is modified by excluding those locations that would correspond to holes formed in the inner peripheral region or the outer peripheral region. By excluding locations in at least one of the outer peripheral region and the inner peripheral region, the strength of the mask in these regions is increased. Several benefits are provided by stronger inner and outer peripheral regions. For example, the mask may be easier to handle during manufacturing or when being applied to a patient without causing damage to the mask.

In another embodiment, the subset of locations is modified by comparing the separation of the holes with minimum and or maximum limits. For example, it may be desirable to assure that no two locations are closer than a minimum value. In some embodiments this is important to assure that the wall thickness, which corresponds to the separation between adjacent holes, is no less than a minimum amount. As discussed above, the minimum value of separation is about 20 microns in one embodiment, thereby providing a wall thickness of no less than about 20 microns.

In another embodiment, the subset of locations is modified and/or the pattern of location is augmented to maintain an optical characteristic of the mask. For example, the optical characteristic may be opacity and the subset of locations may be modified to maintain the opacity of a non-transmissive portion of a mask. In another embodiment, the subset of locations may be modified by equalizing the density of holes in a first region of the body compared with the density of holes in a second region of the body. For example, the locations corresponding to the first and second regions of the non-transmissive portion of the mask may be identified. In one embodiment, the first region and the second region are arcuate regions (e.g., wedges) of substantially equal area. A first areal density of locations (e.g., locations per square inch) is calculated for the locations corresponding to the first region and a second areal density of locations is calculated for the locations corresponding to the second region. In one embodiment, at least one location is added to either the first or the second region based on the comparison of the first and second areal densities. In another embodiment, at least one location is removed based on the comparison of the first and second areal densities.

The subset of locations may be modified to maintain nutrient transport of the mask. In one embodiment, the subset of location is modified to maintain glucose transport.

In a third step, a hole is formed in a body of a mask at locations corresponding to the pattern of locations as modified, augmented, or modified and augmented. The holes are configured to substantially maintain natural nutrient flow from the first layer to the second layer without producing visible diffraction patterns.

IV. Methods of Applying Pinhole Aperture Devices

The various masks discussed herein can be used to improve the vision of a presbyopic patient as well as patient's with other vision problems. The masks discussed herein can be deployed in combination with a LASIK procedure, to eliminate the effects of abrasions, aberrations, and divots in the cornea. It is also believed that the masks disclosed herein can be used to treat patients suffering from macular degeneration, e.g., by directing light rays to unaffected portions of retina, thereby improving the vision of the patient. Whatever treatment is contemplated, more precise alignment of the central region of a mask that has a pin-hole aperture with the line of sight or visual axis of the patient is believed to provide greater clinical benefit to the patient. Other ocular devices that do not require a pin-hole aperture can also benefit from the alignment techniques discussed below. Also, various structures and techniques that can be used to remove an ocular devices are discussed below.

A. Alignment of the Pinhole Aperture with the Patient's Visual Axis

Alignment of the central region of the pinhole aperture 38, in particular, the optical axis 39 of the mask 34 with the visual axis of the eye 10 may be achieved in a variety of ways. In one technique, an optical device employs input from the patient to locate the visual axis in connection with a procedure to implant the mask 34. This technique is described in more detail in U.S. patent application Ser. No. 11/000,562, filed Dec. 1, 2004, the entire contents of which is hereby expressly incorporated by reference herein.

In other embodiments, systems and methods identify one or more visible ocular features that correlate to the line of sight. The one or more visible ocular feature(s) is observed while the mask is being applied to the eye. Alignment using a visible ocular feature enables the mask to perform adequately to increase depth of focus. In some applications, a treatment method enhances the correlation of the visible ocular feature and the line of sight to maintain or improve alignment of the mask axis and the line of sight.

Accurate alignment of the mask is believed to improve the clinical benefit of the mask. However, neither the optical axis of the mask nor the line of sight of the patient is generally visible during the surgical procedures contemplated for implanting masks. However, substantial alignment of the optical axis of the mask and the line of sight may be achieved by aligning a visible feature of the mask with a visible feature of the eye, e.g., a visible ocular feature. As used herein, the term “visible ocular feature” is a broad term that includes features viewable with a viewing aid, such as a surgical microscope or loupes, as well as those visible to the unaided eye. Various methods are discussed below that enhance the accuracy of the placement of the mask using a visible ocular feature. These methods generally involve treating the eye to increase the correlation between the location of the visible ocular feature and the line of sight or to increase the visibility of the ocular feature.

FIG. 48 is a flow chart illustrating one method of aligning a mask with an axis of the eye using a visible ocular feature. The method may include a step of identifying a visible ocular feature, a combination of visible ocular features, or a combination of a visible ocular feature and an optical effect that sufficiently correlate with the location of the line of sight of the eye. In one technique the entrance pupil or other visible ocular feature could be used alone to estimate the location of the line of sight. In another technique, the location of the line of sight can be estimated to be located between, e.g., half-way between, the center of the entrance pupil and the first Purkinje image. Other estimates can be based on a combination of two or more of the first Purkinje image, the second Purkinje image, the third Purkinje image, and the fourth Purkinje image. Other estimates can be based on one or more Purkinje image and one or more other anatomical features. In another technique, the location of the line of sight can be estimated as being located at the center of the pupil if the first Purkinje image is located close to the center of the entrance pupil. A single Purkinje image may provide an adequate estimate of the location of the line of sight if the Purkinje image is generated by a beam having a fixed or a know angle of incidence relative to a surface of the eye. The method may also include a step of identifying a visible feature of the mask to be aligned with a visible ocular feature, as discussed further below.

In a step 1000, an eye is treated to affect or alter, preferably temporarily, a visible ocular feature. In some embodiments, the feature of the eye is altered to increase the correlation of the location of the ocular feature to the line of sight of the eye. In some cases, the treatment of step 1000 enhances the visibility of the ocular feature to the surgeon. The ocular feature may be any suitable feature, such as the pupil or any other feature that correlates or can be altered by a treatment to correlate with the line of sight of the patient. Some techniques involve the alignment of a feature of a mask with the pupil or a portion of the pupil. One technique for enhancing the visibility of the pupil or the correlation of the location of the pupil with the line of sight involves manipulating the size of the pupil, e.g., increasing or decreasing the pupil size.

In connection with the method of FIG. 48, any suitable criteria can be used to confirm alignment of an eye and a mask with a pin-hole aperture. For example, the mask can be considered aligned with the eye when any feature of the mask is aligned with any anatomical landmark on the eye so that an axis passing through the center of the pin-hole aperture is co-linear with or substantially co-linear with an optical axis of the eye, such as the line of sight and an axis passing through the center of the entrance pupil and the center of the eyeball. As used herein, “anatomical landmark” is a broad term that includes an visible ocular feature, such as the center of the entrance pupil, the intersection of the line of sight with a selected corneal layer, the inner periphery of the iris, the outer periphery of the iris, the inner periphery of the sclera, the boundary between the iris and the pupil, the boundary between the iris and the sclera, the location of the first Purkinje image, the location of the second Purkinje image, the location of the third Purkinje image, the location of the fourth Purkinje image, the relative position of any combination of Purkinje images, the combination of the location of a Purkinje image and any other anatomical landmark, and any combination of the foregoing or other anatomical feature.

The pupil size may be decreased by any suitable technique, including pharmacologic manipulation and light manipulation. One agent used in pharmacologic manipulation of pupil size is pilocarpine. Pilocarpine reduces the size of the pupil when applied to the eye. One technique for applying pilocarpine is to inject an effective amount into the eye. Other agents for reducing pupil size include: carbachol, demecarium, isoflurophate, physostigmine, aceclidine, and echothiophate.

Pilocarpine is known to shift the location of the pupil nasally in some cases. This can be problematic for some ocular procedures, e.g., those procedures directed at improving distance vision. The applicant has discovered, however, that such a shift does not significantly reduce the efficacy of the masks described herein.

While the alignment of the masks described herein with the line of sight is not significantly degraded by the use of pilocarpine, an optional step of correcting for the nasal shift of the pupil may be performed.

In one variation, the treatment of the step 1000 involves increasing pupil size. This technique may be more suitable where it is desired to align a visible mask feature near an outer periphery of the mask with the pupil. These techniques are discussed further below.

As discussed above, the treatment of the step 1000 can involve non-pharmacologic techniques for manipulating a visible ocular feature. One non-pharmacologic technique involves the use of light to cause the pupil size to change. For example, a bright light can be directed into the eye to cause the pupil to constrict. This approach may substantially avoid displacement of the pupil that has been observed in connection with some pharmacologic techniques. Light can also be used to increase pupil size. For example, the ambient light can be reduced to cause the pupil to dilate. A dilated pupil may provide some advantages in connection with aligning to a visible mask feature adjacent to an outer periphery of a mask, as discussed below.

In a step 1004, a visible feature of a mask is aligned with the ocular feature identified in connection with step 1000. As discussed above, the mask may have an inner periphery, an outer periphery, and a pin-hole aperture located within the inner periphery. The pin-hole aperture may be centered on a mask axis. Other advantageous mask features discussed above may be included in masks applied by the methods illustrated by FIG. 48. For example, such features may include nutrient transport structures configured to substantially eliminate diffraction patterns, structures configured to substantially prevent nutrient depletion in adjacent corneal tissue, and any other mask feature discussed above in connection with other masks.

One technique involves aligning at least a portion of the inner periphery of a mask with an anatomical landmark. For example, the inner periphery of the mask could be aligned with the inner periphery of the iris. This may be accomplished using unaided vision or a viewing aid, such as loupes or a surgical microscope. The mask could be aligned so that substantially the same spacing is provided between the inner periphery of the mask and the inner periphery of the iris. This technique could be facilitated by making the iris constrict, as discussed above. A viewing aid may be deployed to further assist in aligning the mask to the anatomical landmark. For example, a viewing aid could include a plurality of concentric markings that the surgeon can use to position the mask. Where the inner periphery of the iris is smaller than the inner periphery of the mask, a first concentric marking can be aligned with the inner periphery of the iris and the mask could be positioned so that a second concentric marking is aligned with the inner periphery of the mask. The second concentric marking would be farther from the common center than the first concentric marking in this example.

In another technique, the outer periphery of the mask could be aligned with an anatomical landmark, such as the inner periphery of the iris. This technique could be facilitated by dilating the pupil. This technique may be enhanced by the use of a viewing aid, which could include a plurality of concentric markings, as discussed above. In another technique, the outer periphery of the mask could be aligned with an anatomical landmark, such as the boundary between the iris and the sclera. This technique may be facilitated by the use of a viewing aid, such as a plurality of concentric markings.

In another technique, the mask can be aligned so that substantially the same spacing is provided between the inner periphery of the mask and the inner periphery of the iris. In this technique, the pupil preferably is constricted so that the diameter of the pupil is less than the diameter of the pin-hole aperture.

Alternatively, an artifact can be formed in the mask that gives a visual cue of proper alignment. For example, there could be one or more window portions formed in the mask through which the edge of the pupil could be observed. The window portions could be clear graduations or they could be at least partially opaque regions through which the pupil could be observed. In one technique, the surgeon moves the mask until the pupil can be seen in corresponding window portions on either side of the pin-hole aperture. The window portions enable a surgeon to align a visible ocular feature located beneath a non-transparent section of the mask with a feature of the mask. This arrangement enables alignment without a great amount of pupil constriction, e.g., where the pupil is not fully constricted to a size smaller than the diameter of the inner periphery.

Preferably the alignment of the ocular feature with one or more visible mask features causes the mask axis to be substantially aligned with the line of sight of the eye. “Substantial alignment” of the mask axis with the eye, e.g., with the line of sight of the eye (and similar terms, such as “substantially collinear”) can be said to have been achieved when a patient's vision is improved by the implantation of the mask. In some cases, substantial alignment can be said to have been achieved when the mask axis is within a circle centered on the line of sight and having a radius no more than 5 percent of the radius of the inner periphery of the mask. In some cases, substantial alignment can be said to have been achieved when the mask axis is within a circle centered on the line of sight and having a radius no more than 10 percent of the radius of the inner periphery of the mask. In some cases, substantial alignment can be said to have been achieved when the mask axis is within a circle centered on the line of sight and having a radius no more than 15 percent of the radius of the inner periphery of the mask. In some cases, substantial alignment can be said to have been achieved when the mask axis is within a circle centered on the line of sight and having a radius no more than 20 percent of the radius of the inner periphery of the mask. In some cases, substantial alignment can be said to have been achieved when the mask axis is within a circle centered on the line of sight and having a radius no more than 25 percent of the radius of the inner periphery of the mask. In some cases, substantial alignment can be said to have been achieved when the mask axis is within a circle centered on the line of sight and having a radius no more than 30 percent of the radius of the inner periphery of the mask. As discussed above, the alignment of the mask axis and the line of sight of the patient is believed to enhance the clinical benefit of the mask.

In a step 1008, the mask is applied to the eye of the patient. Preferably the alignment of the optical axis of the mask and the line of sight of the patient is maintained while the mask is applied to the eye of the patient. In some cases, this alignment is maintained by maintaining the alignment of a mask feature, e.g., a visible mask feature, and a pupil feature, e.g., a visible pupil feature. For example, one technique maintains the alignment of at least one of the inner periphery and the outer periphery of the mask and the pupil while the mask is being applied to the eye of the patient.

As discussed above, a variety of techniques are available for applying a mask to the eye of a patient. Any suitable technique of applying a mask may be employed in connection with the method illustrated in FIG. 48. For example, as set forth above in connection with FIGS. 50A-51C, various techniques may be employed to position the mask at different depths or between different layers within the cornea. In particular, in one technique, a corneal flap of suitable depth is hinged open. The depth of the flap is about the outermost 20% of the thickness of the cornea in one technique. In another technique, the depth of the flap is about the outermost 10% of the thickness of the cornea. In another technique, the depth of the flap is about the outermost 5% of the thickness of the cornea. In another technique, the depth of the flap is in the range of about the outermost 5% to about the outermost 10% of the thickness of the cornea. In another technique, the depth of the flap is in the range of about the outermost 5% to about the outermost 20% of the thickness of the cornea. Other depths and ranges are possible for other techniques.

Thereafter, in one technique, the mask is placed on a layer of the cornea such that at least one of the inner periphery and the outer periphery of the mask is at a selected position relative to the pupil. In variations on this technique, other features of the mask may be aligned with other ocular features. Thereafter, the hinged corneal flap is placed over the mask.

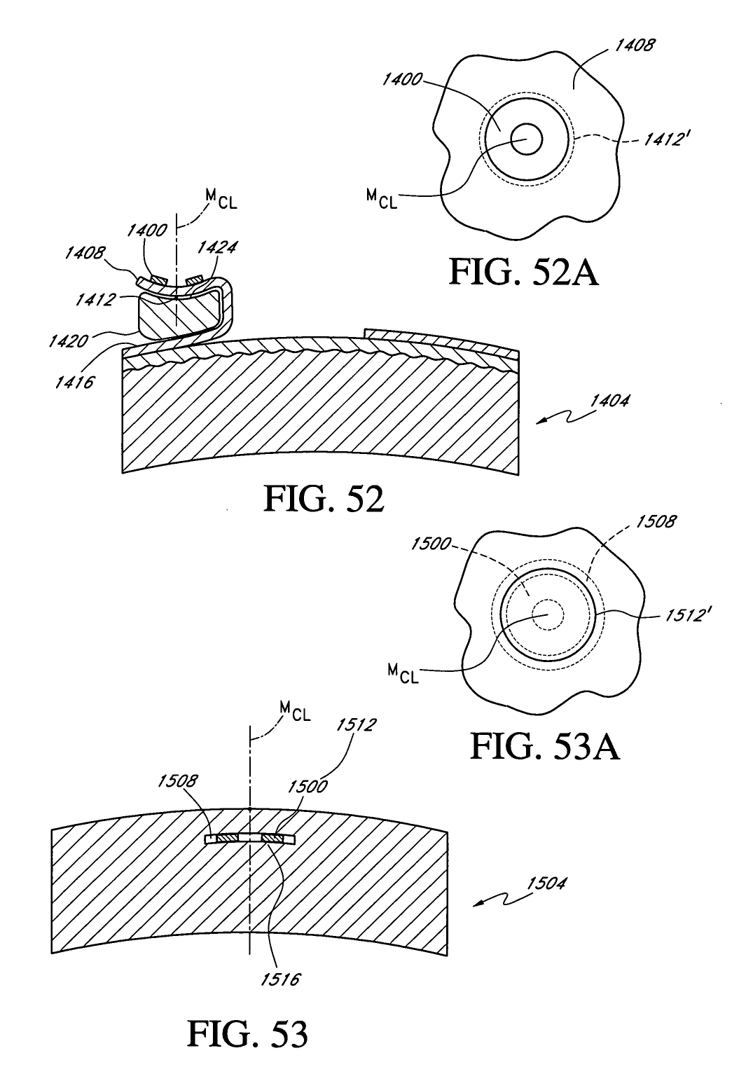

Additional techniques for applying a mask are discussed above in connection with FIGS. 52A-53. These methods may be modified for use in connection with alignment using visible features. These techniques enable the mask to be initially placed on the corneal layer that is lifted from the eye. The initial placement of the mask on the lifted corneal layer may be before or after alignment of a visible ocular feature with a visible mask feature. In some techniques, primary and secondary alignment steps are performed before and after the initial placement of the mask on the lifted corneal layer.

Many additional variations of the foregoing methods are also possible. The alignment methods involving alignment of visible features may be combined with any of the techniques discussed above in connection with optically locating the patient's line of sight. One technique involves removing an epithelial sheet and creating a depression in the Bowman's membrane or in the stroma. Also, the mask can be placed in a channel formed in the cornea, e.g., in or near the top layers of the stroma. Another useful technique for preparing the cornea involves the formation of a pocket within the cornea. These methods related to preparation of the cornea are described in greater detail above.

Some techniques may benefit from the placement of a temporary post-operative covering, such as a contact lens or other covering, over the flap until the flap has healed. In one technique, a covering is placed over the flap until an epithelial sheet adheres to the mask or grows over an exposed layer, such as the Bowman's membrane.

B. Methods of Applying a Mask

Having described method for locating the visual axis of the eye 10 or a visible ocular feature that indicates the location thereof, and for visually marking the visual axis, various methods for applying a mask to the eye will be discussed.

FIG. 49 shows one technique for screening a patient interested in increasing his or her depth of focus. The process begins at step 1100, in which the patient is fitted with soft contact lenses, i.e., a soft contact lens is placed in each of the patient's eyes. If needed, the soft contact lenses may include vision correction. Next, at step 1110, the visual axis of each of the patient's eyes is located as described above. At a step 1120, a mask, such as any of those described above, is placed on the soft contact lenses such that the optical axis of the aperture of the mask is aligned with the visual axis of the eye. In this position, the mask will be located generally concentric with the patient's pupil. In addition, the curvature of the mask should parallel the curvature of the patient's cornea. The process continues at a step 1130, in which the patient is fitted with a second set of soft contact lenses, i.e., a second soft contact lens is placed over the mask in each of the patient's eyes. The second contact lens holds the mask in a substantially constant position. Last, at step 1140, the patient's vision is tested. During testing, it is advisable to check the positioning of the mask to ensure that the optical axis of the aperture of the mask is substantially collinear with the visual axis of the eye. Further details of testing are set forth in U.S. Pat. No. 6,551,424, issued Apr. 29, 2003, incorporated by reference herein in its entirety.

In accordance with a still further embodiment of the invention, a mask is surgically implanted into the eye of a patient interested in increasing his or her depth of focus. For example, a patient may suffer from presbyopia, as discussed above. The mask may be a mask as described herein, similar to those described in the prior art, or a mask combining one or more of these properties. Further, the mask may be configured to correct visual aberrations. To aid the surgeon surgically implanting a mask into a patient's eye, the mask may be pre-rolled or folded for ease of implantation.

The mask may be implanted in several locations. For example, the mask may be implanted underneath the cornea's epithelium sheet, beneath the cornea's Bowman membrane, in the top layer of the cornea's stroma, or in the cornea's stroma. When the mask is placed underneath the cornea's epithelium sheet, removal of the mask requires little more than removal of the cornea's epithelium sheet.

FIGS. 50a through 50c show a mask 1200 inserted underneath an epithelium sheet 1210. In this embodiment, the surgeon first removes the epithelium sheet 1210. For example, as shown in FIG. 50a, the epithelium sheet 1210 may be rolled back. Then, as shown in FIG. 50b, the surgeon creates a depression 1215 in a Bowman's membrane 420 corresponding to the visual axis of the eye. The visual axis of the eye may be located as described above and may be marked by use of the alignment apparatus 1200 or other similar apparatus. The depression 1215 should be of sufficient depth and width to both expose the top layer 1230 of the stroma 1240 and to accommodate the mask 1200. The mask 1200 is then placed in the depression 1215. Because the depression 1215 is located in a position to correspond to the visual axis of the patient's eye, the central axis of the pinhole aperture of the mask 1200 will be substantially collinear with the visual axis of the eye. This will provide the greatest improvement in vision possible with the mask 1200. Last, the epithelium sheet 1210 is placed over the mask 1200. Over time, as shown in FIG. 50c, the epithelium sheet 1210 will grow and adhere to the top layer 1230 of the stroma 1240, as well as the mask 1200 depending, of course, on the composition of the mask 1200. As needed, a contact lens may be placed over the incised cornea to protect the mask.

FIGS. 51a through 51c show a mask 1300 inserted beneath a Bowman's membrane 1320 of an eye. In this embodiment, as shown in FIG. 51a, the surgeon first hinges open the Bowman's membrane 1320. Then, as shown in FIG. 51b, the surgeon creates a depression 1315 in a top layer 1300 of a stroma 1340 corresponding to the visual axis of the eye. The visual axis of the eye may be located as described above and may be marked by any suitable technique, for example using a visible ocular feature or a technique employing patient input. The depression 1315 should be of sufficient depth and width to accommodate the mask 1300. Then, the mask 1300 is placed in the depression 1315. Because the depression 1315 is located in a position to correspond to the visual axis of the patient's eye, the central axis of the pinhole aperture of the mask 1300 will be substantially collinear with the visual axis of the eye. This will provide the greatest improvement in vision possible with the mask 1300. Last, the Bowman's membrane 1320 is placed over the mask 1300. Over time, as shown in FIG. 51c, the epithelium sheet 1310 will grow over the incised area of the Bowman's membrane 1320. As needed, a contact lens may be placed over the incised cornea to protect the mask.

In another embodiment, a mask of sufficient thinness, i.e., less than substantially 20 microns, may be placed underneath epithelium sheet 1210. In another embodiment, an optic mark having a thickness less than about 20 microns may be placed beneath Bowman's membrane 1320 without creating a depression in the top layer of the stroma.

In an alternate method for surgically implanting a mask in the eye of a patient, the mask may be threaded into a channel created in the top layer of the stroma. In this method, a curved channeling tool creates a channel in the top layer of the stroma, the channel being in a plane parallel to the surface of the cornea. The channel is formed in a position corresponding to the visual axis of the eye. The channeling tool either pierces the surface of the cornea or, in the alternative, is inserted via a small superficial radial incision. In the alternative, a laser focusing an ablative beam may create the channel in the top layer of the stroma. In this embodiment, the mask may be a single segment with a break, or it may be two or more segments. In any event, the mask in this embodiment is positioned in the channel and is thereby located so that the central axis of the pinhole aperture formed by the mask is substantially collinear with the patient's visual axis to provide the greatest improvement in the patient's depth of focus.

In another alternate method for surgically implanting a mask in the eye of a patient, the mask may be injected into the top layer of the stroma. In this embodiment, an injection tool with a stop penetrates the surface of the cornea to the specified depth. For example, the injection tool may be a ring of needles capable of producing a mask with a single injection. In the alternative, a channel may first be created in the top layer of the stroma in a position corresponding to the visual axis of the patient. Then, the injector tool may inject the mask into the channel. In this embodiment, the mask may be a pigment, or it may be pieces of pigmented material suspended in a bio-compatible medium. The pigment material may be made of a polymer or, in the alternative, made of a suture material. In any event, the mask injected into the channel is thereby positioned so that the central axis of the pinhole aperture formed by the pigment material is substantially collinear with the visual axis of the patient.

In another method for surgically implanting a mask in the eye of a patient, the mask may be placed beneath the corneal flap created during keratectomy, when the outermost 20% of the cornea is hinged open. As with the implantation methods discussed above, a mask placed beneath the corneal flap created during keratectomy should be substantially aligned with the patient's visual axis, as discussed above, for greatest effect.

In another method for surgically implanting a mask in the eye of a patient, the mask may be aligned with the patient's visual axis and placed in a pocket created in the cornea's stroma.

Further details concerning alignment apparatuses are disclosed in U.S. Provisional Application Serial No. 43/479,129, filed Jun. 17, 2003, incorporated by reference herein in its entirety.

V. Further Methods of Treating a Patient

As discussed above in, various techniques are particularly suited for treating a patient by applying masks such as those disclosed herein to an eye. For example, in some techniques, a visual cue in the form of a projected image for a surgeon is provided during a procedure for applying a mask. In addition, some techniques for treating a patient involve positioning an implant with the aid of a marked reference point. These methods are illustrated by FIGS. 52-53B.

In one method, a patient is treated by placing an implant 1400 in a cornea 1404. A corneal flap 1408 is lifted to expose a surface in the cornea 1404 (e.g., an intracorneal surface). Any suitable tool or technique may be used to lift the corneal flap 1408 to expose a surface in the cornea 1404. For example, a blade (e.g., a microkeratome), a laser or an electrosurgical tool could be used to form a corneal flap. A reference point 1412 on the cornea 1404 is identified. The reference point 1412 thereafter is marked in one technique, as discussed further below. The implant 1400 is positioned on the intracorneal surface. In one embodiment, the flap 1408 is then closed to cover at least a portion of the implant 1400.

The surface of the cornea that is exposed is a stromal surface in one technique. The stromal surface may be on the corneal flap 1408 or on an exposed surface from which the corneal flap 1408 is removed.

The reference point 1412 may be identified in any suitable manner. For example, the alignment devices and methods described above may be used to identify the reference point 1412. In one technique, identifying the reference point 1412 involves illuminating a light spot (e.g., a spot of light formed by all or a discrete portion of radiant energy corresponding to visible light, e.g., red light). As discussed above, the identifying of a reference point may further include placing liquid (e.g., a fluorescein dye or other dye) on the intracorneal surface. Preferably, identifying the reference point 1412 involves alignment using any of the techniques described herein.

As discussed above, various techniques may be used to mark an identified reference point. In one technique the reference point is marked by applying a dye to the cornea or otherwise spreading a material with known reflective properties onto the cornea. As discussed above, the dye may be a substance that interacts with radiant energy to increase the visibility of a marking target or other visual cue. The reference point may be marked by a dye with any suitable tool. The tool is configured so that it bites into a corneal layer, e.g., an anterior layer of the epithelium, and delivers a thin ink line into the corneal layer in one embodiment. The tool may be made sharp to bite into the epithelium. In one application, the tool is configured to deliver the dye as discussed above upon being lightly pressed against the eye. This arrangement is advantageous in that it does not form a larger impression in the eye. In another technique, the reference point may be marked by making an impression (e.g., a physical depression) on a surface of the cornea with or without additional delivery of a dye. In another technique, the reference point may be marked by illuminating a light or other source of radiant energy, e.g., a marking target illuminator and projecting that light onto the cornea (e.g., by projecting a marking target).

Any of the foregoing techniques for marking a reference point may be combined with techniques that make a mark that indicates the location of an axis of the eye, e.g., the visual axis or line-of-sight of the eye. In one technique, a mark indicates the approximate intersection of the visual axis and a surface of the cornea. In another technique, a mark is made approximately radially symmetrically disposed about the intersection of the visual axis and a surface of the cornea.

As discussed above, some techniques involve making a mark on an intracorneal surface. The mark may be made by any suitable technique. In one technique a mark is made by pressing an implement against the instracorneal surface. The implement may form a depression that has a size and shape that facilitate placement of a mask. For example, in one form the implement is configured to form a circular ring (e.g., a thin line of dye, or a physical depression, or both) with a diameter that is slightly larger than the outer diameter of a mask to be implanted. The circular ring can be formed to have a diameter between about 4 mm and about 5 mm. The intracorneal surface is on the corneal flap 1408 in one technique. In another technique, the intracorneal surface is on an exposed surface of the cornea from which the flap was removed. This exposed surface is sometimes referred to as a tissue bed.

In another technique, the corneal flap 1408 is lifted and thereafter is laid on an adjacent surface 1416 of the cornea 1404. In another technique, the corneal flap 1408 is laid on a removable support 1420, such as a sponge. In one technique, the removable support has a surface 1424 that is configured to maintain the native curvature of the corneal flap 1408.

FIG. 52 shows that the marked reference point 1412 is helpful in positioning an implant on an intracorneal surface. In particular, the marked reference point 1412 enables the implant to be positioned with respect to the visual axis of the eye. In the illustrated embodiment, the implant 1400 is positioned so that a centerline of the implant, indicated as M_{CL}, extends through the marked reference point 1412.

FIG. 52A illustrates another technique wherein a reference 1412′ is a ring or other two dimensional mark. In such a case, the implant 1400 may be placed so that an outer edge of the implant and the ring correspond, e.g., such that the ring and the implant 1400 share the same or substantially the same center. Preferably, the ring and the implant 1400 are aligned so that the centerline of the implant M_{CL} is on the line of sight of the eye, as discussed above. The ring is shown in dashed lines because in the illustrated technique, it is formed on the anterior surface of the corneal flap 1408.

In one technique, the corneal flap 1408 is closed by returning the corneal flap 1408 to the cornea 1404 with the implant 1400 on the corneal flap 1408. In another technique, the corneal flap 1408 is closed by returning the corneal flap 1408 to the cornea 1404 over the implant 1400, which previously was placed on the tissue bed (the exposed intracorneal surface).

When the intracorneal surface is a stromal surface, the implant 1400 is placed on the stromal surface. At least a portion of the implant 1400 is covered. In some techniques, the implant 1400 is covered by returning a flap with the implant 1400 thereon to the cornea 1404 to cover the stromal surface. In one technique, the stromal surface is exposed by lifting an epithelial layer to expose stroma. In another technique, the stromal surface is exposed by removing an epithelial layer to expose stroma. In some techniques, an additional step of replacing the epithelial layer to at least partially cover the implant 1400 is performed.

After the flap 1408 is closed to cover at least a portion of the implant 1400, the implant 1400 may be repositioned to some extent in some applications. In one technique, pressure is applied to the implant 1400 to move the implant into alignment with the reference point 1412. The pressure may be applied to the anterior surface of the cornea 1404 proximate an edge of the implant 1400 (e.g., directly above, above and outside a projection of the outer periphery of the implant 1400, or above and inside a projection of the outer periphery of the implant 1400). This may cause the implant to move slightly away from the edge proximate which pressure is applied. In another technique, pressure is applied directly to the implant. The implant 1400 may be repositioned in this manner if the reference point 1412 was marked on the flap 1408 or if the reference point 1412 was marked on the tissue bed. Preferably, pushing is accomplished by inserting a thin tool under the flap or into the pocket and directly moving the inlay.

FIG. 53 shows that a patient may also be treated by a method that positions an implant 1500 in a cornea 1504, e.g., in a corneal pocket 1508. Any suitable tool or technique may be used to create or form the corneal pocket 1508. For example, a blade (e.g., a microkeratome), a laser, or an electrosurgical tool could be used to create or form a pocket in the cornea 1504. A reference point 1512 is identified on the cornea 1504. The reference point may be identified by any suitable technique, such as those discussed herein. The reference point 1512 is marked by any suitable technique, such as those discussed herein. The corneal pocket 1508 is created to expose an intracorneal surface 1516. The corneal pocket 1508 may be created at any suitable depth, for example at a depth within a range of from about 50 microns to about 300 microns from the anterior surface of the cornea 1504. The implant 1500 is positioned on the intracorneal surface 1516. The marked reference point 1512 is helpful in positioning the implant 1500 on the intracorneal surface 1516. The marked reference point 1512 enables the implant 1500 to be positioned with respect to the visual axis of the eye, as discussed above. In the illustrated embodiment, the implant 1500 is positioned so that a centerline M_{CL} of the implant 1500 extends through or adjacent to the marked reference point 1512.

FIG. 53A illustrates another technique wherein a reference 1512′ is a ring or other two dimensional mark. In such case, the implant 1500 may be placed so that an outer edge of the implant and the ring correspond, e.g., such that the ring and the implant 1500 share the same or substantially the same center. Preferably, the ring and the implant 1500 are aligned so that the centerline of the implant M_{CL} is on the line of sight of the eye, as discussed above. The ring is shown in solid lines because in the illustrated embodiment, it is formed on the anterior surface of the cornea 1504 above the pocket 1508.

After the implant 1500 is positioned in the pocket 1508, the implant 1500 may be repositioned to some extent in some applications. In one technique, pressure is applied to the implant 1500 to move the implant into alignment with the reference point 1512. The pressure may be applied to the anterior surface of the cornea 1504 proximate an edge of the implant 1500 (e.g., directly above, above and outside a projection of the outer periphery of the implant 1500, or above and inside a projection of the outer periphery of the implant 1500). This may cause the implant 1500 to move slightly away from the edge at which pressure is applied. In another technique, pressure is applied directly to the implant 1500.

VI. Ocular Devices Having a Locator Structure

As discussed above, various ocular devices may be deployed within the eye, for example within the cornea. Discussed below are various ocular devices that include a locator structure that indicates the location of (e.g., the depth of) the ocular device within the eye when implanted. The structure may also or alternatively be utilized to facilitate removal of the ocular device from the eye as is discussed further below. In some embodiments, the locator structure is an unobtrusive structure that is visible or made visible only to clinical personal during an ocular procedure. The various forms of locator structures discussed below can be used in connection with methods, techniques and procedures for removing a inlay or mask that has been applied in any manner discussed above or in any other suitable manner.

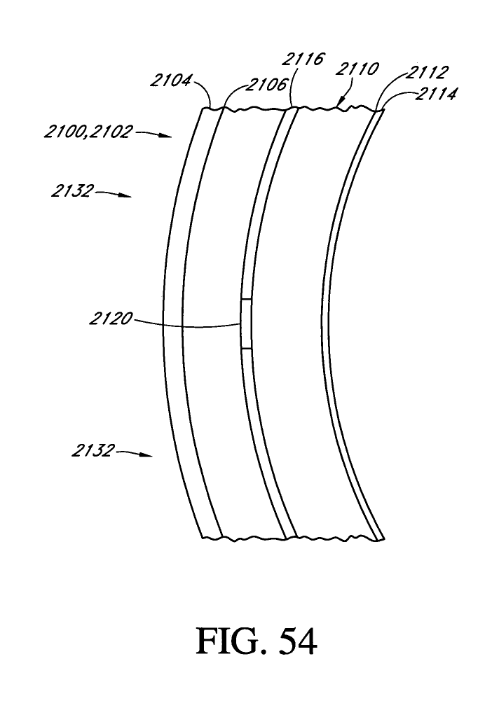

FIG. 54 is a partial side section view of a patient's eye 2100 and more particularly a portion of the cornea 2102 thereof. As illustrated, the cornea 2102 includes an outer epithelium 2104 generally on the order of five cells in thickness with an underlying Bowman's membrane 2106. The stroma 2110 lays on the posterior or rearward side of the Bowman's membrane 2106 and forms approximately 90% of the overall thickness of the cornea 2102. The stroma 2110 is primarily water and collagen and includes a plurality of overlapping and interlocking collagen fibrils that provide a substantially optically transparent and colorless flexible structure, as discussed above. Under the stroma 2110 lies Descemet's membrane 2112. The innermost layer of the cornea is formed by the endothelium 2114, generally a single layer of cells.

FIG. 54 also illustrates that the patient has been provided with a therapeutic implant or inlay 2116 which also defines a central opening 2120. The central opening 2120 can be an aperture sized to provide pin-hole imaging. The inlay 2116 is implanted at a selected layer of the stroma 2110 to provide therapy for one or more vision defects. Depending upon the particular needs of the individual patient, in various embodiments, the inlay 2116 can be of a substantially opaque or transparent nature and can have a generally disc shape, annular shape, or other profile selected for the particular needs of the individual patient.

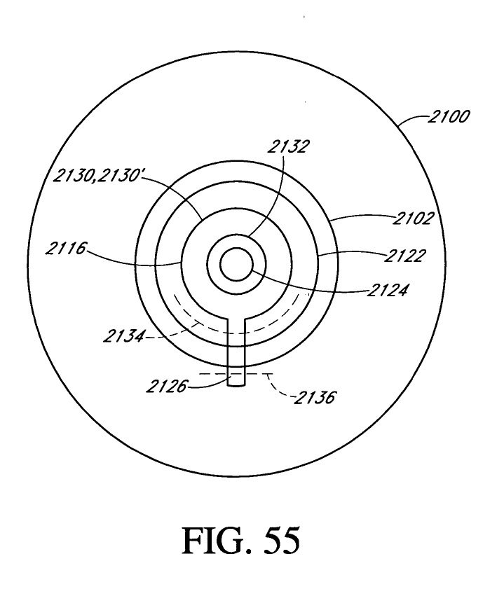

FIG. 55 is a plan view of an anterior or forward portion of a patient's eye 2100 provided with a therapeutic inlay assembly 2130. It should be understood that FIG. 55 is schematic in nature and should not be interpreted as being strictly to scale, however showing generally the eye 2100 including the cornea 2102 and iris 2122 with centrally located pupil 2124. The inlay assembly 2130 is implanted generally centrally in the eye 2100 and in certain embodiments is implanted at a selected layer of the cornea 2102. The inlay 2116 is also configured to at least partially intersect a pupil region 2132 of the eye 2100 along which light enters the eye and is refracted onto the retina.

The inlay assembly 2130 also comprises a locator structure 2126 which is configured to facilitate locating the inlay assembly 2130 after implantation. Normal healing processes result in the incisions being sealed, making the location of the inlay assembly 2130 difficult to find. As discussed further below, the locator structures 2126 make the inlay assembly 2130 easier to find and may be used to facilitate removal of the implant.

In the illustrated embodiment, the locator structure 2126 comprises an elongate tail-like member which extends from a periphery of the inlay 2116. The locator structure 2126 is long enough in the embodiment to extend at least partly beyond the pupil region 2132. As discussed further below, the locator structure 2126 also can be configured to be visible to a surgeon under certain conditions.

Although described primarily with reference to the term “locator structure”, that structure may be utilized to locate the implant, or to locate the implant as well as facilitate removal of the implant, or to facilitate removal of the implant without participating in the process of locating the implant. The locator structure may comprise any of a wide variety of configurations, such as radially outwardly extending flanges, tabs, loops or tethers, depending upon the desired clinical performance. In general, the locator structure 2126 will extend radially outwardly from the periphery of the inlay 2130 for a distance sufficient to extend outside of the patient's line of sight. In certain embodiments, the length of the locator structure 2126 from the periphery of the inlay assembly 2130 will be at least about 25%, in some embodiments at least about 50%, and in other embodiments at least about 75% or 100% or more of the diameter of the inlay assembly 2130.

Although the illustrated locator structure 2126 is configured as a radially outwardly extending tab, having a substantially uniform cross section along its length, and a width of less than about 25% of the diameter of the inlay assembly 2130, any of a variety of alternative structures may be utilized. For example, locator structure 2126 may comprise a tether, such as a single strand or multi-strand filament, extending from the inlay assembly 2130 and provided with a free end, which may be formed into a loop or eye to facilitate grasping by a removal tool. Alternatively, the locator structure 2126 may comprise a strip or band or filament which extends in a closed loop, being attached to the inlay assembly 2130 at 2 points. This provides a loop or handle which may facilitate grasping by a removal tool.

The locator structure 2126 may either be formed integrally with the inlay assembly 2130, or may be formed separately and secured to the inlay assembly 2130 as a separate step. Any of a variety of attachment techniques may be utilized, depending upon the construction materials for the inlay assembly 2130 and the locator structure 2126, such as thermal bonding, adhesive bonding, chemical bonding, interference fit, or other techniques known in the art. Any of a variety of techniques which are known presently in the art for attaching haptics to an intraocular lens may also be used.

Locator structure 2126 may comprise the same material as the inlay assembly 2130, or any of a variety of implantable materials known in the art, such as polypropylene, polyethylene, polyimide, PEEK, Nylon, and a variety of biocompatible metals such as stainless steel, Nitinol or others depending upon the desired performance of the implant.

The design of the locator structure 2126 will also be influenced by the intended post implantation location technique, as will be apparent to those of skill in the art. For example, the locator structures may be configured to be visible under normal direct visualization. Opaque or partially opaque locator structures 2126 may accomplish this objective, such as through the use of metals or polymers having a dye or other constituent which absorbs light in the visible range. However, the cosmetic result may be undesirable, and other location techniques may be preferred as discussed below. An optically transparent locator structure may be located by tactile feedback, such as through the use of a small probe.

Although the present invention is disclosed primarily in the context of an inlay assembly 2130 having only a single locator structure 2126, at least one or two or three locator structures 2126 may be utilized, depending upon the desired clinical performance.

Following implantation, should the patient's condition indicate removal of the inlay assembly 2130, a physician can locate the particular layer of the cornea at which the inlay assembly 2130 is located. This arrangement facilitates easier and quicker access to the locator structure 2126 of the assembly 2130. Also, enabling access outside the pupil region 2132 reduces impact to the optical characteristics within the pupil region 2132 of the eye 2100 by reducing the need to make incisions in this area. In one embodiment, the locator structure 2126 comprises a substantially transparent, thin, and biocompatible material extending outwardly from the periphery of the inlay 2116 so as to overlie a portion of the iris 2122 when applied in the cornea. The locator structure extends beyond the pupil region 2132. The locator structure 2126 of this embodiment is substantially transparent and will therefore be unobtrusive to the patient and not visible under casual observation to others.

For elective removal of the inlay assembly 2130, a physician could form an access incision 2134 towards an outer periphery of the cornea 2102 and at a position outside of the pupil region 2132, aligned with the locator structure 2126. Locating an access incision 2134 outside the pupil region 2132 reduces scarring arising therefrom in the pupil region 2132 and possible deleterious impact on the optical quality of the patient's vision. The physician can then locate the layer at which the inlay assembly 2130 including the inlay 2116 and locator structure 2126 reside either via the access incision 2134 or via a separate location incision 2136. After locating the selected layer or depth at which the inlay assembly 2130 is located, the physician could then grasp the locator structure 2126 and apply a tension force to the locator structure 2126 to withdraw the inlay assembly 2130 through the access incision 2134 or 2136. The appropriate action taken next would depend on the particular condition and indications for the individual patient. In one technique, modification or replacement of the inlay assembly 2130 with a different inlay assembly 2130′ is provided.

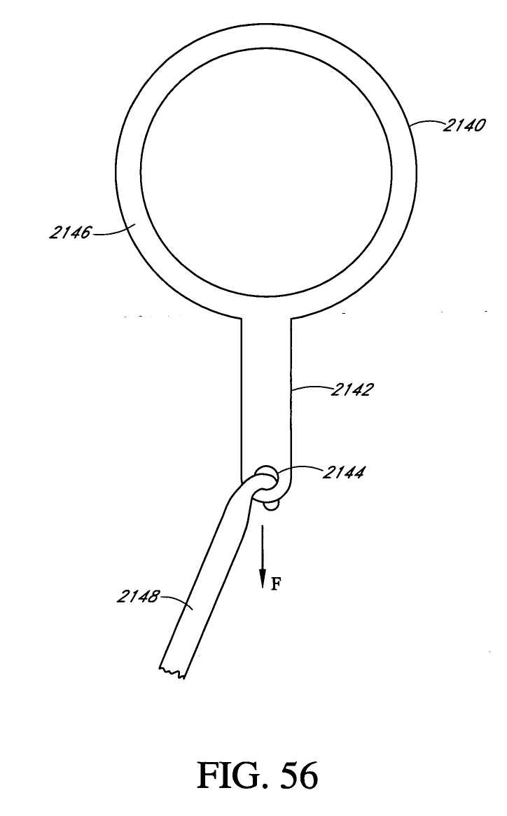

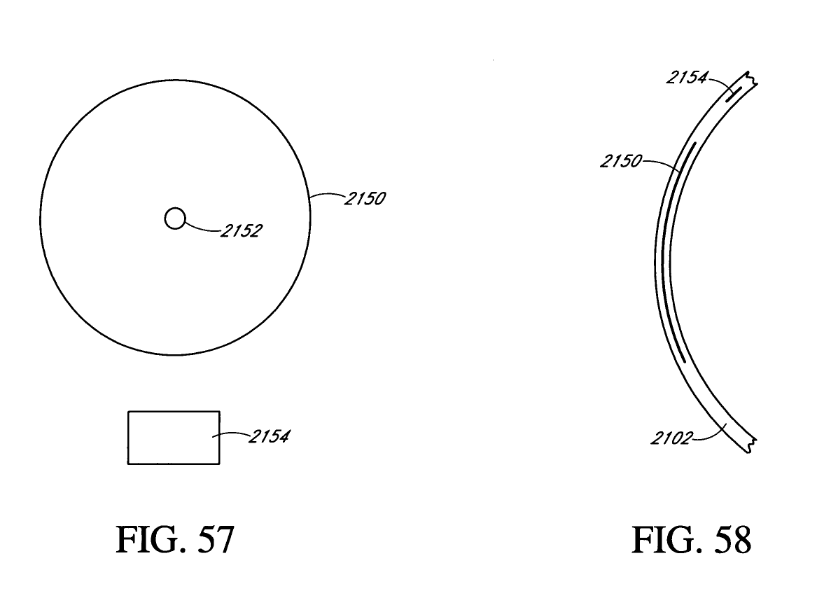

FIG. 56 illustrates another embodiment of an inlay assembly 2140 comprising a locator structure 2142 and a retrieval structure 2144. In general, the retrieval structure 2144 comprises at least one transverse engagement surface 2145 to facilitate engagement by a retrieval instrument. Engagement surface 2145 may be provided in any of a variety of ways. For example, in the embodiment illustrated in FIG. 56, engagement surface 2145 comprises the device distal limit of an aperture formed in the locator structure 2126. In the illustrated embodiment, the retrieval structure 2144 comprises only a single aperture formed in a distal portion of the locator structure 2126. Alternatively, two or three or four or more apertures may be provided in the locator structure 2126.

The retrieval structure 2144 may alternatively be formed by attaching the locator structure 2126 at 2 points to the inlay assembly 2130, to produce a loop or handle configuration. In this configuration, the transverse retrieval surface 2145 is formed on the surface of the locator structure facing the inlay assembly 2130. Any of a variety of alternative retrieval structures 2144 may be provided, depending upon the desired clinical performance, such as providing the locator structure 2126 with texturing, one or more ridges or corrugations, friction enhancing surfaces, or other structure, depending upon the desired cooperation with the complementary surface structures on the desired retrieval tool.

The retrieval structure 2144 can be configured to be engaged by or to receive a retrieval instrument 2148. The retrieval instrument 2148 in one embodiment has at least one transverse engagement surface 2149 for engaging the complementary engagement surface 2145 on the locator structure 2126. In the illustrated embodiment, the engagement surface 2149 is disposed on the inside surface of a generally hook-shaped end of the retrieval tool with which a physician can engage an aperture type retrieval structure 2144. A withdrawing force F can be applied to the retrieval instrument 2148 to extract the inlay assembly 2140. In this embodiment, the inlay assembly 2140 also comprises a therapeutic generally circular shaped body 2146 configured to provide therapy for a patient's vision defect.

FIG. 57 shows another embodiment of a therapeutic ocular inlay 2150 of a generally disc shaped configuration. The inlay 2150 has an aperture 2152, which can be a pinhole aperture, configured to provide pinhole imaging for an eye implanted with the inlay 2150. FIG. 4 also illustrates a separate locator or depth marker 2154 which is configured to facilitate locating the depth of the inlay, but not to participate in the inlay removal process. In application of this embodiment, the inlay 2150 would be implanted in a therapeutic location at least partially overlapping the pupil region 2132 at a selected level of the patient's eye 2100. The locator or marker 2154 would also be implanted at the selected level at a location outside the pupil region 2132. Thus, following implantation and healing processes, a physician could identify and locate the selected level at which the locator or marker 2154 is positioned and after identifying the selected level, proceed at that level to access the inlay 2150, for example for removal and replacement.

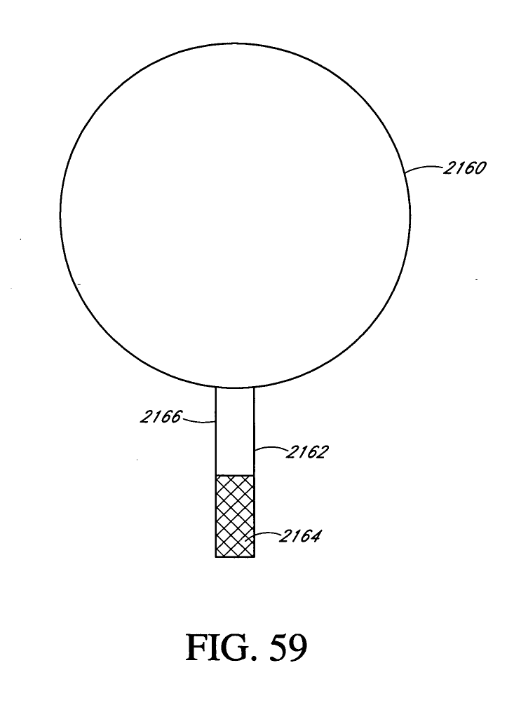

FIG. 59 illustrates a further embodiment of an inlay assembly 2160 with a locator or tail 2162 extending generally outward. In this embodiment, the tail 2162 includes a marker region 2164 positioned at a distal end of the tail 2162 and a transparent region 2166 located at an intermediate or proximal end of the tail 2162. In this embodiment, the marker region 2164 of the tail 2162 is provided with a tinting or coating such that the marker region 2164 exhibits increased contrast with background/underlying eye tissue to facilitate identification and location of the tail 2162 of the inlay assembly 2160. In certain embodiments, the marker region 2164 is formed with selected dye materials such that illumination with electromagnetic radiation of selected wavelengths, in one embodiment ultraviolet light, induces the marker region 2164 to disproportionately luminance or fluoresce in the visible light range to facilitate location of the tail 2162 by a physician or other clinical personnel. Thus, in one embodiment, under routine normal conditions of casual observation, the tail 2162 including the marker region 2164 and transparent region 2166 are substantially invisible. However, under selected observation conditions, in one embodiment, illumination with ultraviolet radiation, the marker region 2164 exhibits enhanced contrast against adjacent tissue. Thus, in one embodiment, the tail 2162 is unobtrusive and substantially invisible under normal casual observation conditions, but, is readily visible under selected artificial viewing conditions to facilitate location of a selected level or depth of the eye in which the inlay assembly 2160 is implanted.

A wide variety of configurations and therapeutic modalities are suitable for use with the various embodiments of ocular devices including a locator structure as described and illustrated herein. Also, any of the features of any one of the various embodiments described hereinabove can be combined with any other embodiment or feature of any other embodiments described hereinabove. Additional details of particular embodiments of ocular inlays which may be advantageously utilized with the invention, e.g., configured with locator structures, are described in greater detail in U.S. application Ser. No. 11/000,562 filed Dec. 1, 2004 and entitled “METHOD OF MAKING AN OCULAR IMPLANT”, in an application with the title “METHOD OF MAKING AN OCULAR IMPLANT” filed Apr. 14, 2005 (Attorney's docket ACUFO.030CP1), and in an application with the title “CORNEAL OPTIC FORMED OF DEGRADATION RESISTANT POLYMER”, all of which are incorporated herein in their entirety by reference.

Although the above disclosed embodiments of the present teachings have shown, described and pointed out the fundamental novel features of the invention as applied to the above-disclosed embodiments, it should be understood that various omissions, substitutions, and changes in the form of the detail of the devices, systems and/or methods illustrated may be made by those skilled in the art without departing from the scope of the present teachings. Consequently, the scope of the invention should not be limited to the foregoing description but should be defined by the appended claims.

What is claimed is:

1. An implantable ocular device comprising:
a therapeutic inlay configured to be implanted at a selected region of a patient's eye at least partially within a pupil region of the eye to provide therapy for at least one vision deficiency; and
a locator structure configured to be implanted within the eye and to extend at least partially outside of the pupil region of the eye to be accessible post-implant from outside the pupil region.

2. The implantable ocular device of claim 1, wherein the selected region comprises a selected layer of a cornea.

3. The implantable ocular device of claim 1, wherein the inlay provides therapy for presbyopia.

4. The implantable ocular device of claim 3, wherein the inlay is at least partially opaque.

5. The implantable ocular device of claim 1, wherein at least part of the locator structure is substantially transparent and colorless.

6. The implantable ocular device of claim 1, wherein the locator structure is connected to the inlay.

7. The implantable ocular device of claim 6, wherein the locator structure further comprises a retrieval structure such that a force applied to the retrieval structure induces the device to be withdrawn from the selected region of the eye.

8. The implantable ocular device of claim 1, wherein at least a portion of the locator structure is provided with an observable tint to provide increased optical contrast with adjacent eye tissue.

9. The implantable ocular device of claim 8, wherein the tint of the locator structure provides increased contrast with adjacent eye tissue under incident electro-magnetic radiation of selected wavelengths outside the visible light range.

10. The implantable ocular device of claim 1, wherein, in an implanted condition, the locator structure is substantially invisible under normal viewing conditions.

11. A method of providing vision therapy comprising;
implanting a first therapeutic inlay in a patient's eye at a selected level, the first inlay having a therapy region configured to provide therapy for at least one vision deficiency; and
implanting a first locator structure at the selected level such that the selected level can be identified at a later time by accessing at least a portion of the first locator structure at a location of the patient's eye outside of the therapy region.

12. The method of claim 11, further comprising
accessing at least a portion of the first locator structure to locate the selected level; and
removing the first therapeutic inlay.

13. The method of claim 12, further comprising:
implanting a second therapeutic inlay in the patient's eye at the selected level, the second inlay having a therapy region configured to provide therapy for at least one vision deficiency; and
implanting a second locator structure at the selected level such that the selected level can be identified at a later time by accessing at least a portion of the locator structure at a location of the patient's eye outside of the therapy region.

14. The method of claim 11, wherein the first locator structure comprises a retrieval structure, the method further comprising:
engaging a instrument with the retrieval structure; and
applying a force through the instrument to the retrieval structure to withdraw the inlay from the selected level.

15. The method of claim 11, further comprising
illuminating the first locator structure with electromagnetic radiation of selected wavelengths to induce at least a portion of the first locator structure to present enhanced contrast against eye tissue adjacent the first locator structure.

16. An inlay for implantation in a cornea of a patient between a first corneal layer and a second corneal layer, the cornea having a pupil region, the inlay comprising:
a mask body having an outer periphery that substantially surrounds a transmissive central portion, the mask body comprising an anterior surface configured to reside adjacent the first corneal layer and a posterior surface configured to reside adjacent the second corneal layer, the mask body configured such that at least the transmissive central portion can be located in the pupil region of the cornea when the inlay is applied; and
a depth marker configured to be positioned between the first and second corneal layers outside the pupil region, the depth marker configured to indicate the location of the mask body.

17. The corneal inlay of claim 16, wherein the depth marker comprises an elongate member having a first end located adjacent to the outer periphery of the mask body and a second end opposite the first end.

18. The corneal inlay of claim 17, wherein the first end is coupled with the mask body.

19. The corneal inlay of claim 17, wherein the depth marker further comprises a visible portion adjacent the second end.

20. The corneal inlay of claim 19, wherein visible portion comprises a fluorescent dye.

21. The corneal inlay of claim 16, wherein the depth marker is configured to indicate the location of at least one of the first and second corneal layers.
